(19) United States
(12) Patent Application Publication
(10) Pub. No.: US 2024/0368703 A1
(43) Pub. Date: Nov. 7, 2024

(54) METHODS FOR PREDICTING PROGRESSION OF BARRETT'S OESOPHAGUS

(71) Applicant: Capsulomics, Inc., Halethorpe, MD (US)

(72) Inventors: Sarah Laun, Baltimore, MD (US); Lisa Kann, Columbia, MD (US); Daniel G. Lunz, Halethorpe, MD (US); Stephen J. Meltzer, Lutherville, MD (US)

(21) Appl. No.: 18/654,593

(22) Filed: May 3, 2024

Related U.S. Application Data
(60) Provisional application No. 63/500,532, filed on May 5, 2023.

(51) Int. Cl. C12Q 1/6886; C12Q 1/686; G16B 20/00; G16H 50/30

(52) U.S. Cl. CPC C12Q 1/6886; C12Q 1/686; G16B 20/00; G16H 50/30; C12Q 2600/118

(57) ABSTRACT

Provided is a method useful in determining the risk of progression of Barrett's esophagus in a subject, comprising providing a cell sample from the esophagus of the subject; detecting one or more biomarkers in the sample using a biochemical assay; determining a parameter associated with the one or more biomarkers; comparing the parameter to at least one predetermined cut-off value indicative of risk of Barrett's esophagus progression; and providing an output based on the comparison.

### CROSS REFERENCE TO RELATED APPLICATIONS

This application claims priority to U.S. Provisional Patent Application No. 63/500,532, filed on May 5, 2023, the disclosure of which is incorporated by reference in its entirety for all purposes.

### FIELD OF THE INVENTION

The present invention relates to a diagnostic method useful in determining the risk of progression of Barrett's oesophagus.

### BACKGROUND OF THE INVENTION

Barrett's esophagus (BE, also referred to Barrett's oesophagus or “BO”) is a condition in which there is an abnormal change in the mucosal cells lining the lower portion of the esophagus, from normal stratified squamous epithelium to simple columnar epithelium with interspersed goblet cells that are normally present only in the small intestine, and large intestine. This change is a premalignant condition because it is associated with a high incidence of further transition to esophageal cancer, and in particular esophageal adenocarcinoma.

Barrett's esophagus is diagnosed by endoscopy, specifically by observing the characteristic appearance of this condition by direct inspection of the lower esophagus, followed by microscopic examination of tissue from the affected area obtained from biopsy. The cells of Barrett's esophagus are classified into four categories: nondysplastic, low-grade dysplasia, high-grade dysplasia, and frank carcinoma. High-grade dysplasia and early stages of adenocarcinoma may be treated by endoscopic resection or radiofrequency ablation. Later stages of cancer may be treated with surgical resection. Those with nondysplastic or low-grade dysplasia are usually managed by annual observation with endoscopy, or treatment with radiofrequency ablation.

The guidelines for the diagnosis and management of Barrett's esophagus can be found in NJ Shaheen et al, (Am J Gastroenterol 2022 Vol. 117 Issue 4 Pages 559-587 Accession Number: 35354777 DOI: 10.14309/ajg.0000000000001680, see https://www.gastrojournal.org/article/S0016-5085 (11) 00084-9/fulltext). This document is hereby incorporated herein by reference. There are equivalent guidelines in the UK, which can be found in Rebecca C Fitzgerald, Massimiliano di Pietro, Krish Ragunath, et al. Management of Barrett's esophagus guidelines on the diagnosis and British Society of Gastroenterology (https://www.bsg.org.uk/wp-content/uploads/2019/12/BSG-guidelines-on-the-diagnosis-and-management-of-Barretts-oesophagus.pdf) updated in the Revised British Society of Gastroenterology recommendations on the diagnosis and management of Barrett's esophagus with low-grade dysplasia.

Esophageal adenocarcinoma is the eighth most common cause of cancer deaths worldwide, and the sixth most lethal cancer in the United States (Wang et al., 2019). The 5-year survival is 20%, and it is responsible for >16,000 deaths per year in the US. Whilst Barrett's esophagus is a known risk factor for adenocarcinoma, only 0.1-3% of cases progress to cancer, and progression remains difficult to identify early or predict in advance. Patients are typically only diagnosed after tumour growth causes symptoms such as difficulty swallowing and weight loss. This, combined with the reliance on endoscopy for diagnosis, means that the condition is only recognised at an advanced stage, where treatment options are limited. Even with regular endoscopic surveillance, any early cases remain undetected 25% of high-grade dysplasia esophageal adenocarcinoma cases occurring within 1 year of endoscopy. Moreover, there remains no consensus on how to manage the majority of Barrett's esophagus patients who are non-dysplastic or indefinite for dysplasia, and these groups remain challenging to risk stratify.

There remains a need for methods which allow the risk of progression of Barrett's esophagus, from lower to higher grades of dysplasia and on to adenocarcinoma, to be determined, and in particular those which allow the prediction of risk at earlier disease stages.

### SUMMARY OF INVENTION

The inventors have surprisingly discovered that the risk of progression of Barrett's esophagus may be determined through the use of a biomarker (or panel of biomarkers) detected in an esophageal sample obtained from a subject. Specifically, the detection and analysis of epigenetic biomarkers in esophageal samples allows more effective surveillance and treatment by predicting risk at an earlier stage. This avoids the need for an invasive endoscopy, and allows earlier detection and prognosis.

The output of this process may include a “risk factor”, stratifying a subject into one of several groups depending on their risk of progression. The use of predictive biomarkers therefore makes surveillance and treatment more efficient by focussing on subjects with the highest risk of progression, and/or advising or making different clinical recommendations (including surgical or therapeutic interventions, and repeat surveillance at determined intervals) based on determined risk.

The analysis of the clinical samples can be done by hand (by a pathologist for example) but it may be more useful to have the analysis conducted using a computer.

Particularly accurate risk determination was possible with the use of a specific panel of biomarkers discussed herein in combination with contextual data about the subject (e.g., patient age), but similarly useful results may be obtained by using additional or alternative biomarkers. As useful results are achievable without age, this feature may be included or omitted from the panels described herein.

Accordingly, in a first aspect, a method useful in determining the risk of progression of Barrett's esophagus in a subject, comprises:
a) providing a cell sample from the esophagus of the subject;
b) detecting one or more biomarkers in the sample using a biochemical assay;
c) determining a parameter associated with the one or more biomarkers from step b);
d) comparing the parameter calculated in step c) to at least one predetermined cut-off value indicative of risk of Barrett's esophagus progression; and
e) providing an output based on the comparison.

In some embodiments, the output comprises a clinical recommendation for the subject. The method may further comprise performing the clinical recommendation. A clinical recommendation may be selected from an endoscopy, drug therapy (e.g. treatment with an NSAID or a PPL, or with an anti-cancer agent), endoscopic resection, endoscopic ablation, radiotherapy, repeat biomarker testing within a specified time-period or a combination thereof. The specified time-period may be 6 months, 1 year, 2 years, 3 years, 4 years or 5 years.

In a second aspect, provided is a method for treating or preventing esophageal dysplasia or esophageal cancer, wherein the subject selected for treatment has been determined to have a risk of progression of Barrett's esophagus by carrying out the method defined in any preceding claim.

In a third aspect, provided is a medicament for use in a method of treating or preventing esophageal dysplasia or esophageal cancer in a subject, wherein the subject selected for treatment has been determined to have a risk of progression of Barrett's esophagus by carrying out the method defined in the first aspect.

Also provided is a method useful in determining the risk of progression of Barrett's esophagus in a subject, comprising:
a) providing detection data on one or more biomarkers detected in a cell sample obtained from the esophagus of the subject, wherein the biomarkers are detected using a biochemical assay;
b) determining a parameter associated with the one or more biomarkers from step a);
c) comparing the parameter calculated in step b) to at least one predetermined cut-off value indicative of risk of Barrett's esophagus progression;
d) providing an output based on the comparison; and optionally
e) selecting the subject to receive and/or administering to the subject a clinical recommendation based on the output provided in step d.

In a further aspect, a computer implemented method useful in determining the risk of progression of Barrett's esophagus in a subject, comprises:
a) receiving detection data on one or more biomarkers detected in a cell sample obtained from the esophagus of the subject, wherein the biomarkers are detected using a biochemical assay;
b) determining a parameter associated with the one or more biomarkers from step a);
c) comparing the parameter calculated in step b) to at least one predetermined cut-off value indicative of risk of Barrett's esophagus progression; and
d) providing an output based on the comparison.

In a yet further aspect, a computer program product stores computer executable instructions for performing the computer implemented steps of the method defined above.

In a further aspect, a method is provided for detecting biomarkers associated with progression of Barrett's oesophagus in a subject and treating the subject, comprising:
a) providing a cell sample from oesophagus of the subject;
b) detecting methylation one or more nucleic acid biomarkers in the sample using a biochemical assay;
c) using a computer algorithm, determining a parameter associated with the detected in step b) methylation of the one or more biomarkers;
d) comparing the parameter calculated in step c) to at least one predetermined cut-off value, wherein the comparison is indicative of risk of Barrett's oesophagus progression in the subject; and
e) performing a Barrett's oesophagus treatment on the subject.

Also provided are treatments for Barrett's oesophagus for use in such a method.

Also provided is a system for determining a risk of progression of Barrett's oesophagus in a subject, comprising:
a) a station for, using a biochemical assay, detecting methylation one or more nucleic acid biomarkers in a sample obtained from the subject;
b) a computer running an algorithm for:
   1) determining a parameter associated with the detected methylation of the one or more biomarkers; and
   2) comparing the parameter calculated in step 1) to at least one predetermined cut-off value, wherein the comparison is indicative of risk of Barrett's oesophagus progression in the subject.

### BRIEF DESCRIPTION OF THE FIGURES

FIG. 1 shows a diagram of the strategy of Esopredict model development and subsequent data validation using the initial retrospective FFPE samples collected from seven clinical sites (n=335). The Esopredict model development branch involves sample processing and analysis of 13 candidate biomarkers and clinical variables (randomly split 2:1) into training (n=147) and test (n=73). The training data undergoes univariate analysis with subsequent logistic regression, and then a multivariable algorithm. The cut points are chosen and generate a 4-tier risk stratification of “low”, “favourable intermediate”/“low moderate”, unfavourable intermediate”/“high moderate” and “high” categories. This risk 4-tier risk stratification is applied to the Esopredict model which then use used on the validation dataset (n=115) consisting of tissue biopsies from 63 non-progressors and 52 progressors, and is also reapplied to the test data set.

FIG. 2 shows a table of the clinical characteristics of patients in the study. The training set (n=147), test set (n=73) ad validation set (n=115) are split into “non-progressors” and “progressors”. The variables considered are Age at Biopsy (yrs); Index-outcome Interval (yrs); Segment Length; Gender; Dysplasia and Institution.

FIG. 3a and b are graphs that show the univariate analysis of methylation-specific biomarkers (FIG. 3a: RUNX3, p16; FIG. 3b: HPP1 and FBN1).

FIG. 4 is a table displaying the logistic regression of RUNX3, p16, HPP1 and FBN1 data, split into “Non-progressor” and “Progressor”, which was evaluated by ROC curves and AUC calculations.

FIG. 5 shows a diagram starting from the initial index biopsy, leading to the follow-up outcome biopsy, whereupon the progression to high-grade dysplasia (HGD)/esophageal adenocarcinoma (EAC) is assessed. At the end of the study, there were n=130 “Progressors” and n=205 “Non-progressors”. The initial retrospective FFPE samples were collected from seven clinical sites (n=335).

FIG. 6 shows the proportions of non-progressors and progressors in each of the four categories of the risk stratification: “low”, “favourable intermediate”, unfavourable intermediate” and “high”. The Negative Predictive Value (NPV)=and patients that progress ≤10 years was 99% are shown for the “low risk category”, and the Positive Predictive Value (PPV) is shown for the “high” risk category.

FIG. 7 shows the Kaplan-Meyer curves of patients that progressed to high-grade dysplasia (HGD) and/or esophageal adenocarcinoma (EAC). Both high to low risk and high to unfavourable intermediate risk were statistically significant (p<0.0001), by LogRank and Wilcoxon tests. The cohort includes both test and validation sets (n=188).

FIG. 8 is a graph that shows the likelihood of “high” vs “unfavourable intermediate”, “high” vs “favourable intermediate” and “high” vs “low” categories progressing to high-grade dysplasia (HGD)/esophageal adenocarcinoma (EAC).

FIG. 9 is a table that shows the prediction of the cumulative progressors based on dysplasia at 2, 5 or 10 years. This includes progressors from the entire study cohort (n=130).

FIG. 10 is a graph that shows that Esopredict identified 87% of “progressors” that progressed to high-grade dysplasia (HGD)/esophageal adenocarcinoma (EAC). This includes progressors from the entire study cohort (n=130).

FIG. 11 is a graph that shows the number of “non-progressors” that Esopredict prevents from unnecessary surveillance, when taking into consideration the biopsy interval in years. Includes “low-risk” patients from the entire cohort (n=85).

FIG. 12 is a graph that shows the number of “non-progressors” that Esopredict prevents from unnecessary surveillance, when taking into consideration the biopsy interval in years, and includes the risk stratification of “low”, “favourable intermediate”, “unfavourable intermediate” and “high” of the “non-progressors”.

FIG. 13 shows that how the Negative Predictive Value (NPV) and Positive Predictive Value (PPV) alters across the four risk categories: “low”, “favourable intermediate”, “unfavourable intermediate” and “high”.

FIG. 14 shows the sensitivity, specificity, PPV and NPV figures produced from Esopredict.

FIG. 15 shows the study enrollment and cohorts for Example 2. Diagram describing the retrospective patient collection cohorts of training, validation, and independent validation. The inclusion criteria are described in the text and in Supplemental FIG. 1.

FIG. 16 shows the model development and training. All of these data represent the training set (n=99). A. Bivariate plot of biomarkers HPP1 and FBN1 with the x-axis showing normalized methylation values of HPP1 and the y-axis showing normalized methylation values of FBNI. Normalized to the internal control b—Actin. B. Coefficients and low and high confidence intervals (CI) of fitting multivariate logistic regression models to the data with standardized predictors including standard error (SE) and z-score (z) C. Predictiveness curve with prevalence-adjusted probability compared to the model score with cut-off values chosen represented by dashed vertical lines. The bold gray line represents the average probability of progression based on prevalence within 5 years. The rug plot across the x-axis shows lines representing patient scores. The red lines indicate a patient that progressed to HGD or EAC, and the black lines indicate a non-progressor (n=99). D. Table reflecting population-adjusted average risk, and estimated number (n) and percent (%) of population-adjusted patients in each risk category. To adjust for prevalence, the number of non-progressors in this cohort was multiplied by a factor of 12 (n=99).

FIG. 17 shows the validation of model. All of these data represent the validation set (n=110). A. Predictiveness curve with prevalence-adjusted probability compared to the model score with locked cut-off values, represented as dashed lines. The bold gray line represents the average probability of progression based on prevalence in 5 years. The rug plot across the x-axis shows lines that represent patient scores, the red lines indicate a patient that progressed to HGD or EAC, and the black lines indicate a non-progressor (n=110). B. A table reflecting population-adjusted average risk, and estimated number (n) and percent (%) of population-adjusted patients in each risk category. To adjust for prevalence, the number of non-progressors in this cohort was multiplied by a factor of 12 (n=110). Also shows is a table listing different risk levels showing the associated risk level compared to average risk of BE patients, the percent risk range, and finally a potential beneficial change in care. C. Estimate the likelihood of progression, i.e., Odds Ratio, within each risk category. Top figure and table: “lower-risk” (low-+low moderate-risks combined) compared to “higher-risk” (high moderate- and high-risks combined). Bottom table: Comparing risk categories to the low-risk category, including lowest confidence level (LCL) and upper confidence level (UCL). Due to prevalence, the number of non-progressors in this cohort was represented by a multiplication of 12 (n=110).

FIG. 18 shows the performance of Esopredict. All of these data represent the validation set (n=110). A. Table with the number of patients (n) in each risk level and overall separated by non-progressor and progressor. 1.1 Includes all patients, 1.2 Includes only patients with an index biopsy (i.e., assayed) of NDBE (non-dysplastic). B. Includes only patients that had an index biopsy (i.e., assayed) of NDBE (non-dysplastic), and table with the number of patients (n), population-adjusted percent of patients, and the average prevalence-adjusted risk percent across each category and risk level in only those patients that had an index biopsy (i.e., assayed) of NDBE (non-dysplastic).

FIG. 19 shows model performance in the independent validation. A. Boxplots of the Esopredict scores (y-axis) of non-progressor (black, n=70) and progressor patients in the first validation (red, n=40) for the two left plots, the right plot (represents independent validation patients that progressed >5 years (n=31). The bold line represents the median score, a the box represents the first and third quartiles. B. Analysis of variance (ANOVA) of the 3 patient populations: non-progressor (n=70), progressor (n=40) from the validation, and progressions with outcome biopsies >5 years (n=31).

FIG. 20 illustrates example steps for determining the risk of progression of Barrett's oesophagus.

FIG. 21 illustrates a schematic diagram of a computer system for performing the method.

### DETAILED DESCRIPTION

The present invention is useful in determining the risk of progression of Barrett's esophagus in a subject.

Barrett's esophagus (also referred to as Barrett's oesophagus, “BO” or “BE”) is a condition in which there is an abnormal, metaplastic change in mucosal cells lining the esophagus, believed to result from chronic acid exposure due to reflux. During the disease, mucosal cells transition from stratified squamous epithelium to simple columnar epithelium with interspersed goblet cells typical of the intestine. It is considered a premalignant condition, associated with further development to esophageal cancer.

The severity or stage of Barrett's esophagus may be measured by endoscopic surveillance to monitor the degree of dysplasia. As used herein, the term “dysplasia” means a pre-cancerous stage in Barrett's esophagus, wherein the cells develop abnormal features, but do not have the ability to spread to other sites. Dysplasia advances from non-dysplastic (ND), to indefinite for dysplasia (IFD), to low-grade dysplasia (LGD) and finally high-grade dysplasia (HD). Barrett's esophagus as used herein may be associated with any of these degrees of dysplasia.

Barrett's esophagus with high grade dysplasia may alternatively be referred to as Stage O esophageal cancer.

The severity or stage of Barrett's esophagus may also be characterised by Prague stage (Sharma et al., Gastroenterology. 2006;131:1392-9. Epub 2006 Aug. 16, incorporated by reference herein). “Prague stage” incorporates two values: a measurement of the difference between a lower bound defined proximal margin of cardial folds and the proximal margin of the circumferential Barrett's segment (″C), and the difference between the lower bound and the proximal margin of the longest tongue of Barrett's tongue (M). Prague stage may typically be diagnosed by endoscopic means.

In some embodiments, the method is useful in determining the risk of progression of Barrett's esophagus to esophageal cancer. Such a method may be viewed as determining the risk of a subject developing esophageal cancer. “Esophageal cancer” refers to a malignant tumour affecting or comprising cells of the esophagus. In some embodiments, the esophageal cancer is esophageal adenocarcinoma. The esophageal cancer may be of the upper, middle, or lower esophagus, or may affect the gastroesophageal junction.

As used herein, “progression” of Barrett's esophagus may refer to an increase in the presence, frequency, or severity of one or more symptoms of Barrett's esophagus over time. For example, progression may refer to an increase in dysplasia stage, and/or Prague stage.

The methods aim to determine the risk of disease progression in a subject. Alternatively, it may be viewed as determining the probability that a subject's disease will progress. When performed at an early stage, this may be viewed as predicting the likelihood of disease progression in a subject.

In some embodiments, a subject with Barrett's esophagus has “progressed” if their condition increases in dysplasia stage and/or develops into esophageal cancer. For example, progression has occurred if a subject previously diagnosed with or characterised as having Barrett's esophagus characterised as non-dysplastic, low grade dysplasia, or indefinite for dysplasia is subsequently diagnosed with or characterised as having high grade dysplasia and/or esophageal cancer. The previous and subsequent diagnosis may be separated by a defined period, for example of 1, 1.5, 2, 2.5, 3, 3.5, 4, 4.5, or 5 or more years.

In some embodiments, the “risk of progression” may be the risk of disease progression within a defined period. For example, the defined period may be 1, 1.5, 2, 2.5, 3, 3.5, 4, 4.5, 5, 6, 7, 8, 9, 10 or more years. For example, subjects whose disease progresses within 1, 2, 3, 4 or 5 years may be referred to as “progressors”, whilst those who do not progress within 1, 2, 3, 4 or 5 years may be called “non-progressors”.

In particular embodiments, subjects whose disease progresses within 5 years may be referred to as “progressors”, whilst those who do not progress within 5 years may be called “non-progressors”.

In other embodiments, subjects whose disease progresses within 3 years may be referred to as “progressors”, whilst those who do not progress within 3 years may be called “non-progressors”. In further embodiments, subjects whose disease progresses within 10 years may be referred to as “progressors”, whilst those who do not progress within 10 years may be called “non-progressors”.

In some embodiments, subjects whose disease progresses within 1 year may be referred to as “progressors”, whilst those who do not progress within 4.5 years be called “non-progressors”.

In some embodiments, the subject has been identified or diagnosed as having Barrett's esophagus. In some embodiments, the subject is diagnosed with Barrett's esophagus characterised as having no dysplasia, reactive atypia, indefinite for dysplasia, low grade dysplasia, or high grade dysplasia. In other embodiments, the subject is diagnosed with Barrett's esophagus characterised as having no dysplasia, reactive atypia, indefinite for dysplasia, or low grade dysplasia. In other embodiments, the subject is diagnosed with Barrett's esophagus characterised as having no dysplasia, or reactive atypia. In other embodiments, the subject is diagnosed with Barrett's esophagus characterised as not having high grade dysplasia or as not having low or high grade dysplasia.

A subject may be identified as having a Prague stage which is at least C1, at least M1, at least C2, at least M2, at least C3, at least M3, at least C4, at least M4, at least C5, at least M5, at least C6, at least M6, at least C7, at least M7, at least C1 or M3 or any combination thereof. More preferably, the Prague state is at least C1 or M3 (i.e. either, both, or more), which is recognised as useful in diagnosis in the clinical guidelines. A subject may exhibit dysplasia or have no dysplasia. As the method is capable of early detection, a subject may be at an early Prague stage (e.g., at least C1 or M3), or at an early stage of dysplasia (e.g. ND, IFD or LGD, but not HGD). In some embodiments, the subject has advanced stage Barrett's esophagus, which may be characterised by dysplasia (e.g., LG or HD) or late Prague stage (e.g., at least C2 or C3).

In other embodiments, the subject has not been identified as having Barrett's esophagus but may have been identified as being at risk of developing Barrett's esophagus and/or esophageal cancer. They may also have been selected as part of routine screening. For example, all males over 55 might be screened routinely using the method of the invention.

In some embodiments, the subject has one or more risk factors for esophageal cancer and/or progression of Barret's esophagus. These may be selected from: a) being age 55 or over; b) being a man; c) being a smoker; d) being an alcohol drinker; e) having gastresophageal reflux disease; f) being obese; g) suffering from achalasia; h) having a history of certain other cancers; and/or i) suffering from Tylosis or Plummer-Vinson syndrome. In some embodiments of the present invention, subjects with gastresophageal reflux disease may be selected for the diagnostic test of the invention. Those patients may be at particularly high risk of developing Barrett's esophagus, and of disease progression.

In some embodiments, the subject has, is suspected of having, has been identified as being at risk of developing, or has been identified as having one or more risk factors for, esophageal cancer.

The methods involve providing one or more cell samples from the esophagus of the subject. As used herein, a cell sample is any sample which comprises a cell or cell-derived product. In some embodiments, the sample comprises a tissue, cell, a population of cells, or a cell-derived product, e.g., a vesicle. A sample may comprise one or more cells from the surface of the esophagus. Any cell sampling device that collects cells from the surface of the esophagus may be used in a method of the invention. Whilst samples can be obtained through endoscopic methods, and/or can be resection or biopsy samples, such as a Formalin-Fixed Paraffin-Embedded (FFPE) biopsy, in some embodiments, the sample is a non-endoscopic sample. These may be less invasive than endoscopic or surgical resection methods. In some embodiments, the sample is provided by esophageal brushing or by a swallowable sponge device.

It may be beneficial to use a “pill on a string” device, where the sample is provided by retrieving a swallowable device from the subject that has been swallowed by the subject, wherein the device comprises an abrasive material configured to collect the cells. One such cell sampling device is EsoCheck. Another suitable cell sampling device is Cytosponge. Cytosponge consists of a small gelatin capsule. This contains a compressed spherical polyester sponge which is attached to string. The capsule is swallowed and after 5 minutes the capsule dissolves allowing the sponge to expand. Using the string, a nurse then pulls the sponge from the stomach through the esophagus and out of the mouth. As it travels up the esophagus it collects cells including some from Barrett's if it is present.

As used herein, a “biomarker” is any molecule, gene, or characteristic (e.g. epigenetic modification) associated with a pathological process, i.e., risk of progression of Barrett's esophagus. Any biomarker for risk of progression of Barrett's esophagus may be used in a method of the invention. It may be appropriate to use more than one biomarker in a method of the invention, such as by using a combination or a panel of biomarkers. Biomarkers include but are not limited to protein biomarkers, expression or RNA biomarkers, genetic or DNA biomarkers, or epigenetic biomarkers (e.g. DNA methylation biomarkers).

The biomarker is detected using a biochemical assay. Suitable biochemical assays are known, and they are able to differentiate cells that comprise the biomarker and cells that do not comprise the biomarker. Biomarkers may be qualitative (i.e., “present” or “absent”) or quantitative (represented as e.g. a percentage, frequency of occurrence, or normalised amount). In some embodiments, the biomarker can be detected by immunological means, such that the assay is able to differentiate between cells that express the biomarker and cells that do not express the biomarker.

In preferred embodiments, the biomarker may be a methylation biomarker, i.e. methylation of a nucleic acid encoding a gene, such that it is detectable by DNA methylation analysis. Advantageously, as epigenetic remodelling and altered methylation is one of the earliest and most pervasive DNA changes during disease progression, this allows earlier risk prediction and determination of future risk, even at stages before neoplasia or dysplasia have occurred or can be detected.

DNA methylation may be detected by any technique known in the art or described herein.

In some embodiments, a methylation biomarker is or incorporates methylation within the promoter region, the coding region, and/or the non-coding region of one or more genes. These genes are indicative of risk of progression.

In some embodiments, the biomarkers comprise methylation of one or more genes selected from:
Cadherin 13, H-cadherin (heart) (CDH13);
tachykinin-1 (TAC1);
nel-like 1 (NELL1);
A-kinase anchoring protein 12 (AKAP12);
soma tostatin (SST);
transmembrane protein with EGF-like and two follistatin-like domains (HPP1);
CDKN2a, cyclin dependent kinase inhibitor 2a (p16);
runt-related transcription factor 3 (RUNX3);
ATP binding cassette subfamily B member 1 (ABCB1);
Bone morphogenic protein 3 (BMP3);
Collagen type XXIII alpha 1 chain (COL23A1);
Fibrillin 1 (FBN1);
Fatty acid desaturase 1 (FADS1); and
PR Domain zinc finger protein 2 (PRDM2)

In some embodiments, the biomarkers comprise methylation of one or more genes selected from: p16, RUNX3, HPP1, FBN1, mVIM, mCCNA1, or p53.

In some embodiments, the biomarkers comprise methylation of one or more (e.g., 2, 3 or 4) genes selected from: P16, RUNX3, HPP1, and FBN1. In some embodiments, the biomarkers comprise methylation of FBN1 and of one, two, or three genes selected from P16, RUNX3, HPP1. In preferred embodiments, the biomarkers comprise methylation of all four genes P16, RUNX3, HPP1, and FBN1. These four biomarkers may be combined with one or more further clinical factors associated with the subject, and in particular age, to provide a robust determination of risk of progression.

In some embodiments, the biomarkers comprising methylation of one or more of genes P16, RUNX3, HPP1, and LBN1, may further comprise one or more (e.g. 2, 3, 4, 5, 6, 7, 8, 9, or 10,) additional biomarkers selected from p53; tachykinin-1 (TAC1); nel-like 1 (NELL1); A-kinase anchoring protein 12 (AKAP12); soma tostatin (SST); ATP binding cassette subfamily B member 1 (ABCB1); Bone morphogenic protein 3 (BMP3); Collagen type XXIII alpha 1 chain (COL23A1); Fibrillin 1 (FBN1); Fatty acid desaturase 1 (FADS1); Cadherin 13 (CDH13), and PR domain zinc finger protein 2 (PRDM2). In preferred embodiments, the additional gene is p53 and/or a tumour micro-environment marker. These additional biomarkers may be methylation status of the relevant gene, but alternative biomarkers are also contemplated, including next generation sequencing, Immunohistochemistry, fluorescence in situ hybridization, extracellular DNA, and single cell analysis.

The biomarker is detected using a biochemical assay. Suitable biochemical assays are known, and they are able to differentiate cells that comprise the biomarker and cells that do not comprise the biomarker. Preferably, the biomarker can be detected by DNA methylation analysis, for example bisulphite sequencing. In such a method, a methylation biomarker may be detected by 1) providing a methylated DNA sample extracted from the cell sample, 2) performing bisulphite conversion on methylated DNA so as to convert all unmethylated cytosine residues to uracil, and 3) detecting the pattern of methylation in the sample. This detection may be performed through any suitable technique, including polymerase chain reaction (PCR)-based techniques (e.g. quantitative methylation specific PCR (qMSP), digital methylation specific PCR (dPCR), or droplet digital methylation specific PCR (ddPCR)), sequencing methylation specific DNA (NGS), pyrosequencing, direct sequencing, and microarray-based methods. Alternatively, a “next generation sequencing” technique may be employed, for example pyrosequencing, whole genome bisulfite sequencing, reduced representation bisulfite sequencing, or methylated DNA immunoprecipitation sequencing. Suitable methods and procedures for these techniques will be known to the skilled person.

A mixture of biomarkers may be used, with different detection techniques. For example, one or more methylation biomarkers may be combined with one or more protein biomarkers detected via e.g. immunohistochemistry, mRNA analysis, or any other suitable means. Certain biomarkers can be detected via DNA methylation analysis, PCR-based techniques (in particular where the biomarker is an expression biomarker), or immunohistochemistry. Suitable techniques will be known to the skilled person and include fluorescence techniques and staining techniques. The detecting of the biomarker may comprise immunohistochemically staining a tissue section of the sample.

Biomarker detection may involve normalising levels or occurrence of the biomarker to a reference. The reference may be an external reference, for example a matched cell sample or a standard reference. Alternatively, the biomarker may be normalised to a reference determined from the same sample (“normalised to self”).

The present invention involves determining a parameter associated with the detected biomarkers. This may be determined using a computer algorithm.

In some embodiments, the algorithm is a machine learning algorithm, such as a supervised learning algorithm.

In some embodiments, the algorithm is a regression algorithm, for example a linear regression algorithm, or a logistic regression algorithm. In some embodiments, the algorithm is a least absolute shrinkage and selection operator (LASSO) algorithm.

Alternative algorithms for use in the invention include random forest, Xboost, time to event/time series analysis, cox regression, decision trees, and machine learning algorithms described herein.

An algorithm may be capable of learning or optimisation through “training”, or may be “pre-trained” (i.e. have undergone training, and unable to further learn/optimise). During training, an algorithm is provided labelled training data, comprising examples associated with subjects for whom biomarkers (inputs) and progression outcome (outputs) are known. The algorithm infers a function from the paired inputs and outputs, which can be used to map new unseen examples. The form of the function is in part determined by the design of algorithm (e.g., a linear regression algorithm will produce a linear regression function). When inferring a function, the algorithm may differentially “weight” the inputs so as to provide a more accurate prediction of output, as determined by statistical test. The algorithm may iteratively adjust the weights of the inputs to refine the function. The accuracy of the learned function may be evaluated through the use of paired verification data. Once a suitably accurate learned function has been determined, training may be stopped-the use of such a learned function as “fixed code” without further learning may be referred to as a “pre-trained algorithm”.

An algorithm may be tested or validated prior to use. This may involve presenting the algorithm with a validation data set comprising paired inputs-outputs. This data set should be different to the training data set. The accuracy of the algorithm may be determined by comparing the output of the algorithm to the known outputs for the validation data set.

The algorithmic determination may incorporate additional contextual data about the subject, such as additional clinical factors associated with the subject. These factors may increase the predictive accuracy of the parameter. Relevant clinical factors include age, gender (e.g., being male), presence or absence of esophageal dysplasia, dysplasia grade, Barrett's esophagus segment length, BMI or obesity, lifestyle factors (e.g. being a smoker or alcohol drinker), having gastresophageal reflux disease (GERD), suffering from achalasia, having a history of certain other cancers; and/or suffering from Tylosis or Plummer-Vinson syndrome. In preferred embodiments, the additional factors include age and optionally one or more of gender (e.g., being male), the presence/absence of dysplasia, dysplasia grade, and Barrett's esophagus segment length.

The determined parameter may be represented as a numeric output, for example as a value on a scale of 0 to 1, whereby 1 is the highest risk, and 0 the lowest. The bounds of this scale may correspond to clinical situations, for example a score of 0 may represent a 18 yr old with no methylation, and a score of 1 may represent 95 yr old with 2× methylation in all genes. In some embodiments, the output is a value on a scale of −1 to 1, whereby 1 is the highest risk, and −1 the lowest.

The skilled person will readily appreciate that such a numeric output may be presented on any arbitrary scale, or as for example a percentage, without meaningfully changing the underlying parameter.

The methods involve comparing the calculated parameter to at least one predetermined cut-off value indicative of risk of Barrett's esophagus progression, and producing an output based on that comparison.

A “predetermined cut-off value” may be determined through any means, for example through analysis of biomarkers in subjects for whom progression is known. The algorithms described above may determine or include the least one predetermined cut-off value.

For example, in some embodiments, an “upper predetermined cut-off value” is used which defines subjects with a high risk of progression. A subject is assigned a “high risk” of progression if the calculated parameter is equal to or above the upper predetermined cut-off value. Such a subject is predicted to have a high probability of disease progression, and this may affect clinical recommendations. For example, high risk subjects may be selected or prioritised for surgical or therapeutic treatment. Subjects equal to or below the upper predetermined cut-off value may be assigned to a “non-high risk” group. Such a subject may be deprioritised for treatment, and/or referred for one-off or recurrent surveillance endoscopy or repeat biomarker sampling.

Predetermined cut-off values may be optimised for specificity. This means that the value is selected such that it correctly categorises “progressors” or “non-progressors” as defined herein. This may be expressed as a percentage.

In some embodiments, the upper cut-off value is selected such that at least about 65%, at least about 70%, at least about 75%, at least about 80%, at least about 85%, at least about 90%, at least about 95%, or at least 99% of subjects in the high risk group are progressors. Corresponding, the cut-off value may be selected so that no more than about 45%, no more than about 30%, no more than about 20%, no more than about 15%, no more than about 10%, no more than about 10%, no more than about 5%, or no more than about 1% subjects in the high risk group are non-progressors. This minimises false positives i.e., non-progressors labelled as high risk (type II error). For example, the upper cut-off value may be selected so that about 90% or more subjects in the high risk group are progressors.

The non high risk group incorporates a variety of risk profiles, making clinical recommendations challenging. It may therefore be further stratified through the use of additional cut-off values. For example, in some embodiments, a “lower predetermined cut-off value” is used to define subjects with a low risk of progression. The lower predetermined cut-off value is lower than the higher predetermined cut-off value.

A subject is assigned a “low risk” of progression if the calculated parameter is equal to or below the lower predetermined cut-off value. As these subjects have low probability of disease progression, they may be given less invasive clinical recommendations. For example, the frequency of surveillance endoscopies may be reduced or eliminated.

In some embodiments, the lower cut-off value is selected such that at least about 65%, at least about 70%, at least about 75%, at least about 80%, at least about 85%, at least about 90%, at least about 95%, or at least 99% of subjects in the lower risk group are non-progressors. Corresponding, the cut-off value may be selected so that no more than about 45%, no more than about 30%, no more than about 20%, no more than about 15%, no more than about 10%, no more than about 10%, no more than about 5%, or no more than about 1% subjects in the low risk group are progressors. This minimises false negatives i.e., progressors labelled as low risk (type I error). For example, the lower cut-off value may be selected so that about 90% or more subjects in the low risk group are non-progressors.

Subjects not assigned to the low or high risk groups may be assigned to a “intermediate risk” group. Clinical recommendations for such a group will lie between the extremes of the other two groups. However, it may be advantageous to stratify this group further into “unfavourable intermediate risk” and/or “favourable intermediate risk” through the use of an additional “intermediate predetermined cut-off value”. The intermediate predetermined cut-off value is between the upper and lower predetermined cut-off values. The intermediate cut-off value is selected so that about more subjects in the unfavourable intermediate risk group are predicted to undergo disease progression than those in the favourable intermediate risk group.

A subject is assigned an unfavourable intermediate risk of progression if the calculated parameter is equal to or above the intermediate predetermined cut-off value, and equal to or below the upper predetermined cut-off value. Subjects with an unfavourable intermediate risk of progression may benefit from a clinical recommendation comprising increased frequency of surveillance endoscopy and/or selected or prioritised for surgical or therapeutic treatment e.g., for adenocarcinoma.

A subject is assigned a favourable intermediate risk of progression if the calculated parameter is equal to or above the lower predetermined cut-off value, and equal to or below the intermediate predetermined cut-off value. Subjects with a favourable intermediate risk of progression may benefit from a clinical recommendation comprising following the standard of care for Barrett's esophagus (e.g., drug therapy with NSAIDs or PPL) maintaining or increasing the frequency of surveillance endoscopy, but is unlikely to be selected or prioritised for surgical or therapeutic treatment e.g. for adenocarcinoma.

In some embodiments, the lower cut-off value is selected such that at least about 65%, at least about 70%, at least about 75%, at least about 80%, at least about 85%, at least about 90%, at least about 95%, or at least 99% of subjects in the lower risk group are non-progressors. Corresponding, the cut-off value may be selected so that no more than about 45%, no more than about 30%, no more than about 20%, no more than about 15%, no more than about 10%, no more than about 10%, no more than about 5%, or no more than about 1% subjects in the low risk group are progressors. This minimises false negatives i.e., progressors labelled as low risk (type I error). For example, the lower cut-off value may be selected so that about 90% or more subjects in the low risk group are non-progressors.

In some embodiments, an intermediate cut-off value is selected to preserve 75% specificity. For example, the intermediate cut-off value may be selected so that at least about 60%, at least about 65%, at least about 70%, at least about 75%, or at least about 80% of subjects meeting or exceeding the cut-off value are progressors. Corresponding, the cut-off value may be selected so that no more than about 40%, no more than about 35%, no more than about 30%, no more than about 25%, or no more than about 20% of subjects meeting or exceeding the cut-off value are non-progressors.

The skilled person will readily appreciate that any number of further intermediate predetermined cut-off values may be used to divide subjects into further risk categories. Indeed, n predetermined cut-off values may be used to define n+1 risk categories.

The output of the methods herein may comprise one or more factor associated with the subject including any of the determined parameter (either “raw” or “transformed” e.g., conversion to a percentage scale or expressed as a colour, such as blue, green, red or amber.), information about the presence or levels of one or more biomarker, a determined risk, and/or a risk category as described above.

In certain embodiments, the output provides information which may be used to make a clinical recommendation. Such information may include any factor or information which, alone or in combination with further information, aides or allows a clinician to make a suitable clinical recommendation. This may include further information about the subject's condition e.g. dysplasia stage or Prague stage. The information may be in the form of a print-out or display detailing the patient's risk classification and/or biomarker data. This may be beneficial in guiding a physician in selecting a suitable schedule of care.

In some embodiments, the output comprises a clinical recommendation for the subject. In further embodiments, the method comprises performing the clinical recommendation. A clinical recommendation may comprise one or more intervention selected from: retesting using the methods of the invention, endoscopic surveillance, surgical treatment, and/or therapeutic treatment. A clinical recommendation may comprise a schedule of interventions independently selected from this list, optionally within a defined time period or with a defined frequency. This may be beneficial in guiding a physician in selecting a suitable schedule of care.

In some embodiments, the method comprises administering a treatment to the subject, or recommending that a treatment is administered to the subject. As used herein, a “treatment” includes any intervention with the aim of curing, reversing, preventing, slowing the progression, or preventing the spread of a disease or symptom. Relevant surgical treatments may include ablation or esophageal resection. Surgical treatments may be performed via an endoscope. The treatment is preferably selected from an endoscopy, drug therapy, endoscopic resection, endoscopic ablation, repeat biomarker testing within a specified time-period or a combination thereof. The output may be any recommendation listed in the clinical guidelines for diagnosis/management of Barrett's esophagus. These will be known to the skilled person and are referenced herein (and incorporated herein by reference). Further treatments include proton pump inhibitors (PPIs), histamine receptor blockers, lifestyle changes (to reduce acid reflux e.g. eat small meals during the day, do not eat for 2-3 hours before bedtime, elevate the head by 10 to 20 cm during sleep, avoidance of spicy/fatty foods, avoidance of alcohol, weight loss, and/or smoking cessation), multipolar electrocoagulation, or cryotherapy. Treatments may be surgical, for example fundoplication, resection (e.g. endoscopic mucosal resection), ablation (e.g. radiofrequency ablation), or oesophagectomy.

A therapeutic treatment may include drug therapy. Drug therapy may be intended to reverse or halt further progression of Barrett's esophagus or its symptoms (as with NSAID and/or PPI). Alternatively, the drug therapy may include an anti-cancer agent intended to treat, prevent, or slow the progression of esophageal cancer. Such drugs are known to the skilled person and referenced in the clinical guidelines. Therapeutic treatments may also include radiotherapy, chemotherapy, targeted therapy (e.g., therapy with an inhibitor, for example antibody therapy), or a combination thereof.

Treatments may also include recommendations for endoscopic surveillance or retesting using the methods of the invention. The recommendation may include a specified time-period in or after which surveillance or retesting is to be performed. This time period may be 6 months, 1 year, 2 years, 3 years, 4 years or 5 years. The recommendation may also provide a schedule for repeated surveillance or retesting at a given frequency. This frequency may be every 1 month, 2 months, 3 months, 4 months, 5 months, 6 months, 1 year, 2 years, 3 years, 4 years or 5 years. The schedule may include one, two, three, four, five, six, seven, eight, nine, ten or more repeats. The schedule may be indefinite.

The output of the test method of the invention may include or be the diagnosis of a particular stage of Barret's esophagus in the subject, for example Prague stage or extent of dysplasia.

In some embodiments, the method may be useful for treating or preventing esophageal dysplasia or esophageal cancer, wherein the subject selected for treatment has been determined to have a risk of progression of Barrett's esophagus by carrying out the method as described above.

In some embodiments, there is a medicament which is useful in the method of treating or preventing esophageal dysplasia or esophageal cancer in a subject, wherein the subject selected for treatment has been determined to have a risk of progression of Barrett's esophagus by carrying out the method described above. In some embodiments, the medicament is an NSAID or a PPL, or an anti-cancer agent.

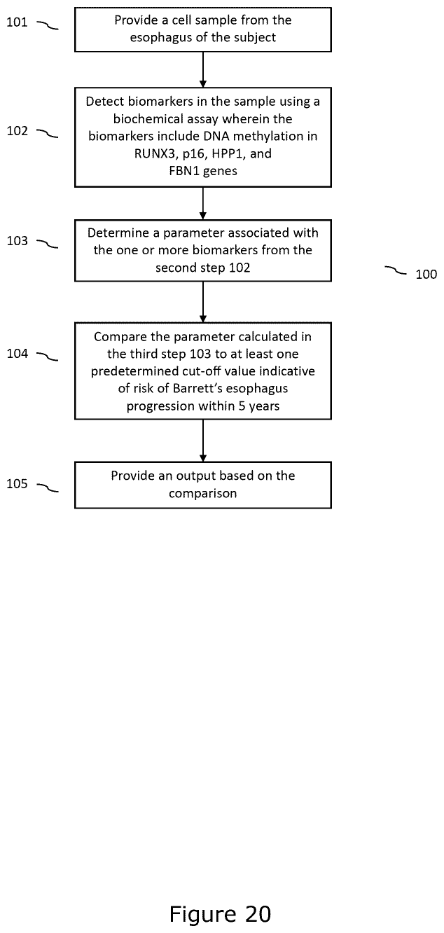

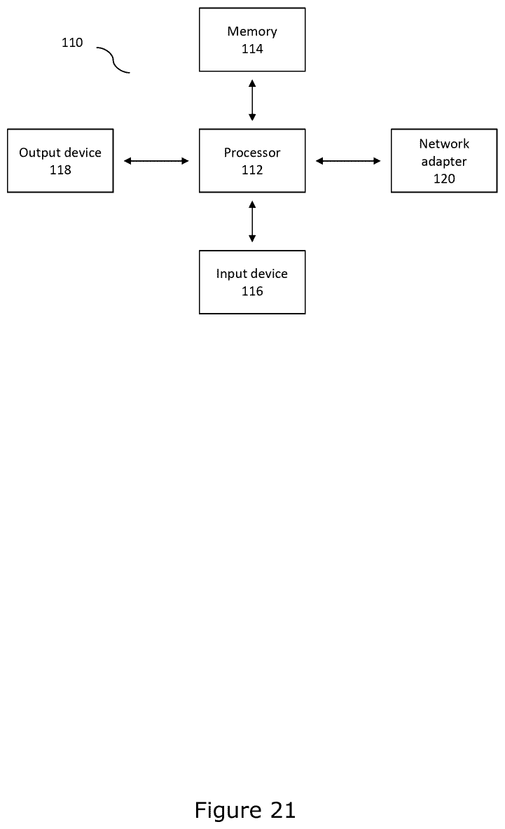

In some embodiments, any of the above described methods may be implemented using a computer. FIG. 20 illustrates a schematic block diagram of a computer system 110 suitable for implementing a method as described above. The computer system 110 comprises one or more processors 112 in communication with memory 114. The memory 114 is an example of a computer readable storage medium.

In some examples, the one or more processors 112 may be in communication with one or more input devices 116 and/or one or more output devices 118. The various components of the computer system 110 may be implemented using generic means for computing known in the art. For example, an input device 116 may comprise a keyboard or mouse and an output device 118 may comprise a monitor or display, and an audio output device such as a speaker. In some examples, the computer 110 may further comprise network access circuitry, such as a modem or network adaptor 120, to provide access to a network.

It will be appreciated that, in some examples, one or more of the various hardware units may be integrated with one another. For example, if the computer system 110 is a tablet computer or cellular telephone, a display of the device 110 may provide both the display of the output device 118 and a touch sensor providing the input device 116.

All cited documents are incorporated herein by reference.

As used herein, the open “comprises” encompasses closed “consists”, and all reference to “comprising” may be replaced by the narrower “consisting”.

### EXAMPLES

The following example illustrate the invention.

### Overview

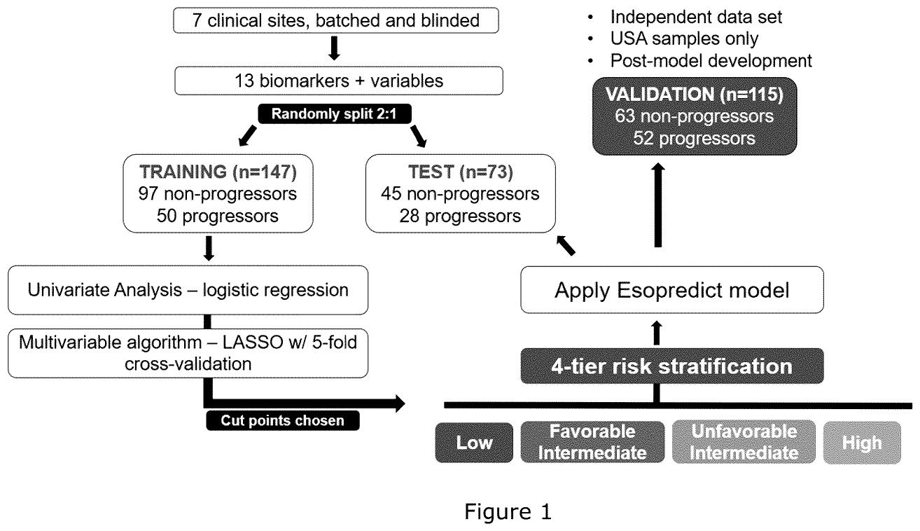

The Esopredict model was developed using training and test data to produce a 4-tier risk stratification assay, then applied to validate independent data and thus risk stratify Barrett's esophagus patients (FIG. 1).

### Methods

### Cell Sample Collection

Barrett's esophagus cell samples were collected from patients (via endoscopic or non-endoscopic methods) from 7 clinical sites that were batched and blinded (n=335). Retrospective formalin-fixed paraffin-embedded (FFPE) Barrett's esophagus cell samples were subsequently received by the laboratory.

### DNA Extraction from FFPE

### Dissolving Paraffin Wax

1. Mix Deparaffinization solution to ensure the solution is homogeneous.
2. Add 300 μl of Deparaffinization solution to each sample tube, vortex vigorously for 10 secs. Flick down or quick spin tube contents to ensure liquid and sample are at the bottom of the tube.
3. Incubate at 56° C. for 3 mins, 1000 rpms in the thermomixer.
4. Allow samples to cool to room temperature for 5 mins.
5. Check to determine if sample appears waxy or solid after cooling. This indicates paraffin is still intact. If needed add an additional 300 μl of deparaffinization solution and repeat incubation at 56° C. for 3 mins, 1000 rpms in the thermomixer and allow to cool to room temperature for 5 mins.
6. If the sample still appears waxy/solid after cooling, add an additional final 300 μl of deparaffinization solution (900 μl total) and repeat the incubation at 56° C. for 3 mins, 1000 rpms in the thermomixer. Allow sample to cool to room temperature for 5 mins.
7 In the rare case that the sample is still waxy or solid after cooling, this sample will require further division across tube to ensure the paraffin is completely digested. To do so the sample is reheated at 56° C. for 3 mins 1000 rpms in the thermomixer.
8. The contents of the tube are then split 50:50 across two tubes at 450 μl each. Then an additional 300 μl of deparaffinization solution is added and repeat incubation at 56° C. for 3 mins. Allow sample to cool to room temperature for 5 mins.

### Tissue Digestion with Proteinase K

1. Generate a master mix of PK solution for 100 μl/sample as the following: 25 μl of Buffer FTB, 55 μl RNase-free water, and 20 μl Proteinase K. Example if 10 samples (X11) 250 ul of Buffer FTB, 550 μl of RNase-free water, and 200 μl of Proteinase K. Distribute 100 μl to each sample.
2. Add 100 μl of master-mix PK solution to each sample. Vortex to mix. Centrifuge briefly to bring down any tissue or solution on the tube wall or under the cap.
3. Incubate at 56° C., and 1000 rpm in Thermomixer overnight 12-16 hours.
4. Remove tubes from thermomixer and set thermomixer to 90° C., 0 rpms.
5. Incubate the samples in Thermomixer at 90° C., 0 rpms for 1 hour.
6. Take out samples and set the thermomixer to 65° C., 450 rpms.
7. Quickly spin to remove drops from inside the lid.
8. Carefully remove and discard the upper blue phase in each tube. Keep the lower aqueous lysate. Be sure to not mix the layers, it that occurs, spin down and try again. Use a pipette-tip-step-down method with larger pipette at first and then come back to the sample with a smaller-pipette tip to remove the last of blue liquid. Re-centrifuge if needed to ensure all sample is transferred and separation is consistent.
9. Add 150 μl RNase-free water, vortex to mix and quick spin.
10. Add 2 μl RNase A, vortex to mix, and incubate at room temperature for 2 mins.
11. Briefly spin to remove drops from inside the lid.
12. Add 20 μl Proteinase K, vortex, and incubate in Thermomixer at 65° C. and 450 rpms for 15 mins.
13. Briefly spin to remove drops from inside the lid.

### DNA Isolation

1. As the second Proteinase K incubation is occurring, take out columns with collection tubes from 4° C. and label accordingly. Each sample gets 1 column and collection tube regardless of how many tubes the sample was dived in for the deparaffination experiment. Example: Deparaffination occurred across 4 tubes from the same sample number and label, only one column labelled with the same sample and label is needed.
2. Add 250 μl of Buffer AL to each sample and mix by inverting the tube 5-10×.
3. Add 250 μl ethanol (96-100%) to each sample and mix by inverting the tube 5-10×.
4. Briefly spin to remove drops from inside the lid and ensure all of the sample is in the bottom of the tube. Line up tubes to indicate transfer from initial sample tubes to the corresponding pre-labelled columns.
5. Carefully transfer up to 500 μl lysate to QIAamp MinElute column (in a 2 ml collection tube) without wetting the rim.
6. Centrifuge at 14,600 rpm (15,000× g) for 30 secs.
7. Discard the flow through and replace collection tube.
2.1.3.8. If multiple tubes were used for deparaffination step for the same sample, transfer the additional lysate (500 μl maximum volume) from the same sample to the sample column ensure verification of tube transfer on the worksheet and centrifuge at 14,600 rpm (15,000× g) for 1 min.
9. Discard flow-through and reuse the collection tube.
10. Repeat as necessary until all the lysate is through the column.
11. Carefully open column and add 500 μl Buffer AW1 without wetting the rim for each sample.
12. Close the lid and centrifuge at 14,600 rpm (15,000× g) for 30 secs.
13. Discard the flow-through and reuse the collection tube.
14. Take care when removing the collection tube and column from the rotor to ensure that the flow through, which contains alcohol, does not come in contact with the column.
15. Carefully open the column and add 500 μl Buffer AW2 without wetting the rim.
16. Close the lid and centrifuge at 14,600 rpm (15, 000× g) for 30 secs.
17. Discard the flow-through and reuse the collection tube.
18. Carefully open column lid and add 250 μl ethanol (96-100%) to each sample.
19. Centrifuge at 14,600 rpm (15, 000× g) for 30 secs.
20. Discard flow-through and collection tube. Place spin column into a new 2 ml collection tube.
21. Centrifuge at max speed (close to 20,000× g) for 3 mins. to dry the membrane completely.
22. Set up new pre-labelled 1.5 mL DNA low-bind tubes so that the corresponding columns of the same sample number and label are lined up.
23. Place the QIAamp MinEIute column the corresponding tube with. Column lid will be closed, microcentrifuge tube lid will be open.
24. Carefully open the lid of the column and apply 44 μl of buffer ATE to the centre of the membrane.
25. Close the lid and incubate at room temperature for 10 mins.
26. Centrifuge at max speed (close to 20,000× g) for 1 min with the open lid of the centrifuge tubes pointing inwards to the centre of the rotor.
27. Re-apply the 44 μl eluate to the same column by collecting the 44 μl from the microcentrifuge tube and placing in the centre of the column. Incubate on benchtop for 5 additional mins.
28. Centrifuge at max speed (close to 20,000× g) for 1 min with the open lid of the centrifuge tubes pointing inwards to the centre of the rotor.
29. Remove column, and carefully secure the lids of each sample tube.
30. Proceed directly to Qubit HS dsDNA quantification or store DNA in pre-labelled box at −20° C.

### Bisulfite Conversion

1. Calculate the amount of DNA for bisulfite treatment, using the concentration of DNA calculated using Qubit assay.
2. Add 20 μl of DNA to 1.5 ml tube ensuring secondary verification of tube transfer.
3. Add 130 μl of Lightning Conversion Reagent to 20 μl of DNA.
4. Incubate samples at 98° C. for 8 mins in the Thermomixer, at 0 RPM.
5. Incubate samples at 98° C. for 8 mins in the Thermomixer, at 0 RPM.
6. Store samples at 4° C. for up to 20 hours. Or skip this step if continuing.
7. Add 600 μl of M-Binding Buffer to a Zymo-Spin IC Column and place into the collection tube.
8. Load the DNA sample into the column ensuring tube transfer verification, and mix by inverting the column several times.
9. Centrifuge at max speed of 15,060 rpm (>10,000× g) for 30 secs. Discard flow-through.
10. Add 100 μl M-Wash Buffer to the column.
11. Centrifuge at max speed of 15,060 rpm (>10,000× g) for 30 secs.
12. Add 200 μl M-Desulphonation Buffer to the column.
13. Let stand at room temperature for 15 mins.
14. Centrifuge at full speed for 30 secs.
15. Add 200 μl M-Wash Buffer to the column.
16. Centrifuge at max speed of 15,060 rpm (>10,000× g) for secs.
17. Add 200 μl M-Wash Buffer to the column.
18. Centrifuge at max speed of 15,060 rpm (>10,000× g) for 30 secs.
19. Place in a new 1.5 ml microcentrifuge tube labelled with sample ID and ensuring secondary verification of tube transfer.
20. Add 140 μl molecular-grade water. Incubate at room temperature for 5 mins.
21. Centrifuge at max speed of 15,060 rpm (>10,000× g) for 60 secs.
22. Store at 4° C. for up to 1 day, or at −20° C. for up to 6 months.
23. DNA is ready for use in methylation-specific PCR.

### Methylation Analysis

Methylation levels of the candidate biomarkers (CDH13; TAC1; NELL1; AKAP12; SST; HPP1; p16; RUNX3, ABCB1; BMP3; COL23A1; FBN1; FADS1; PRDM2) were determined by quantitative methylation specific PCR (qMSP). The methylation level of the gene was normalised.

Normalized methylation values, along with patient age is ran through logistic regression algorithm. Score between 0-1 is assigned with 0 being the lowest risk of progression and 1 being the highest risk of progression. Three pre-determined cut-offs used to develop a 4-tier stratification. The output, along with the risk-tier is provided to GI physician to aid in determining management of the patient condition.

### Model Development

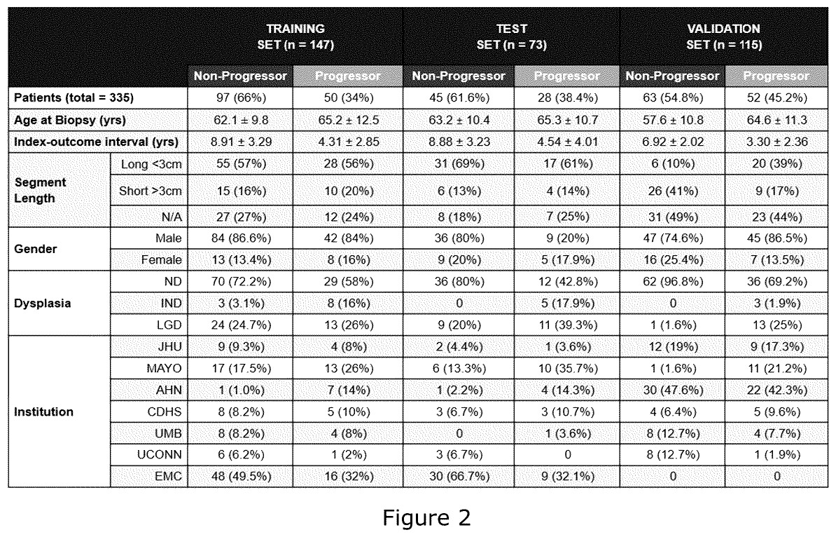

Processing of FFPE samples from 335 patients, in combination with analysis of 4 candidate biomarkers (HPP1, p16, RUNX3 and FBN1) and clinical variables (age, gender and dysplasia) (see FIG. 2). The samples were randomly split at a 2:1 ratio. The training data set (n=147) consisted of tissue biopsies from 97 non-progressors and 50 progressors. The test data set (n=73) consisted of tissue biopsies from 45 non-progressors and 28 progressors.

### Univariable Analysis

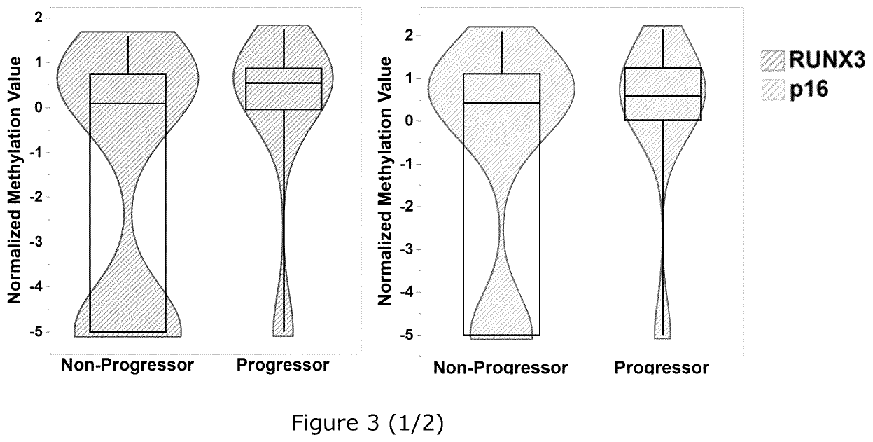

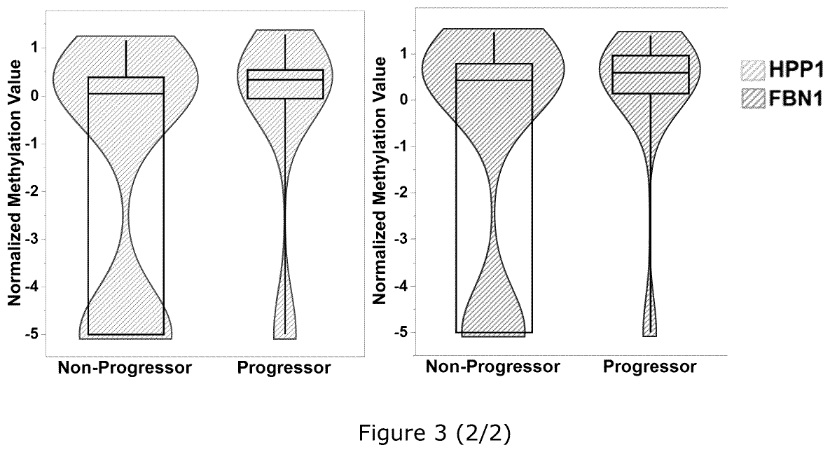

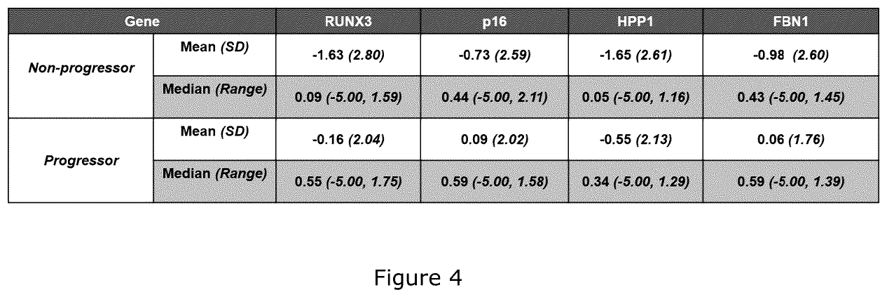

Univariable analysis was performed upon the training set data (n=147), using the 4 biomarkers, along with patient age, index-outcome interval, segment length, gender, dysplasia and institution (FIG. 3). Logistic regression was evaluated by ROC curves and AUC calculations (FIG. 4).

### Multivariable Algorithm

A multivariable algorithm was subsequently applied to the training set, using LASSO (Least Absolute Shrinkage and Selection Operators) regression and logistic regression, wherein the parameters were chosen via 5-fold cross validation.

### Cut-Offs for Risk Categories

Based upon the multimodal distribution of the risk score, cut-offs for risk categories were identified. Wherein specificity was preserved at 90% for a high cut-off point, and at 75% for an intermediate cut-off point.

The lower cut-off (defining the boundary between low and favourable intermediate risk categories) was −0.47796. The intermediate cut-off (defining the boundary between favourable intermediate/low moderate and unfavourable intermediate/high moderate risk categories) was 0.12. The upper cut-off (defining the boundary between unfavourable intermediate and high risk categories) was 0.21767.

### Validation

Separate samples (from the USA) were collected and processed using the Esopredict model trained by the 4 biomarkers and patient age, for post-model development. The independent validation data set (n=115) consisted of tissue biopsies from 63 non-progressors and 52 progressors.

### 4-Tier Risk Stratification

The Esopredict model development, data from the test set and from the validation set were then combined to produce a 4-tier risk stratification. The 4 tiers are:
Low
Favourable Intermediate/low moderate
Unfavourable Intermediate/high moderate
High

### Results

### Proportions of Non-Progressors and Progressors

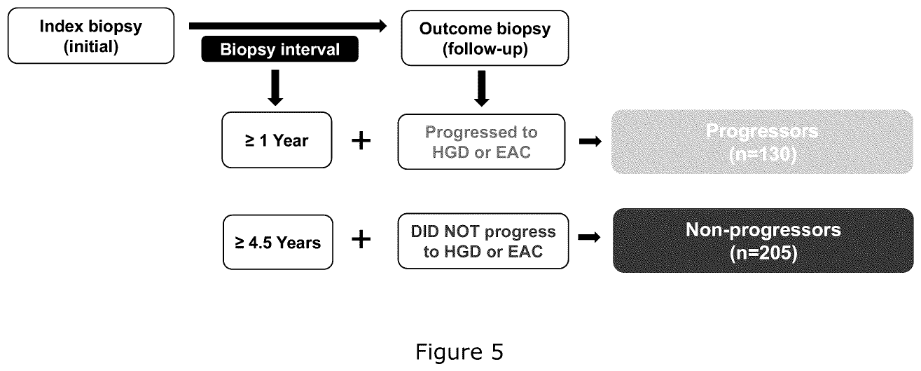

Esopredict was used to generate proportions of non-progressors and progressors (cohort includes test and validation sets) which were then assigned to one of the four risk categories: “low”, “favourable intermediate”, “unfavourable intermediate”, and “high” (Table 1) (FIG. 5).

TABLE 1
Proportions of non-progressors and progressors.

| Risk Category | Non-progressors | Progressors | Total |
|---|---|---|---|
| Low | 50 | 12 | 62 |
| Favorable intermediate | 41 | 26 | 67 |
| Unfavorable intermediate | 14 | 22 | 36 |
| High | 3 | 20 | 23 |

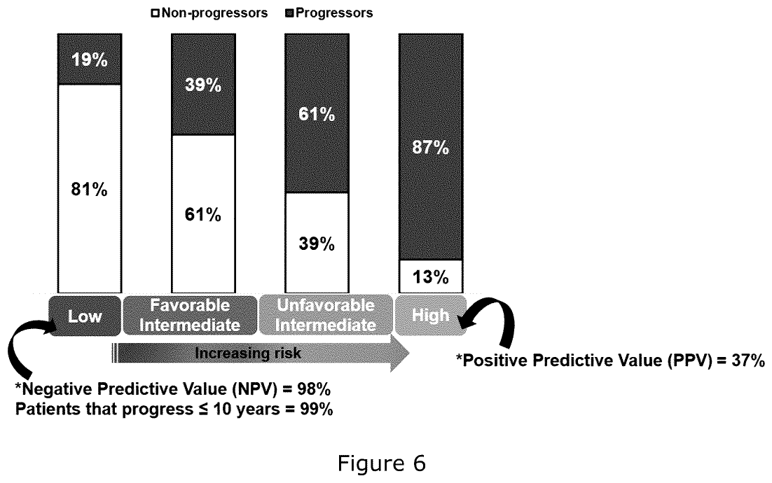

In the “low” risk category, the Negative Predictive Value (NPV) was 98% and patients that progress ≤10 years was 99%, whereas in the “high” risk category the Positive Predictive Value (PPV) was 37% (FIG. 6). The percentage of progressors increased from 19% in the “low” risk category to “87” in the “high” risk category.

### Kaplan-Meier and Hazard Ratios

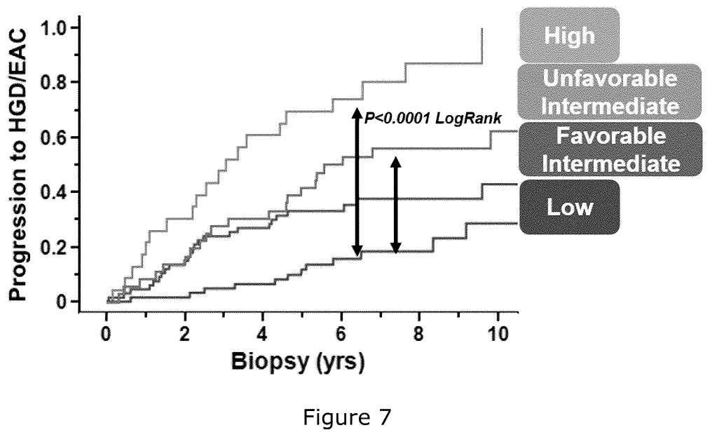

Comparison of the “high” to “low” risk categories and “unfavourable intermediate” to “low” risk categories were both statistically significant (p<0001), using LogRank and Wilcoxon tests (FIG. 7). The results can be seen in Table 2.

TABLE 2

| Reference to high risk category | Hazard Ratio (95% CI) | ChiSquare |
|---|---|---|
| High vs. Low | 8.25 (4.01, 17.0) | 0.0001 |
| High vs. Favourable Intermediate | 3.48 (1.93, 6.28) | 0.0001 |
| High vs. Unfavourable Intermediate | 2.24 (1.21, 4.15) | 0.010 |

### Odds Ratio

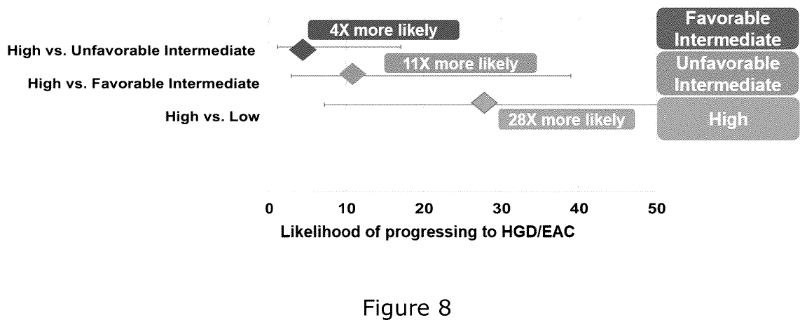

When compared, “high” vs “unfavourable intermediate”, “high” vs “favourable intermediate” and “high” vs “low” categories showed a 4×, 11× and 28× likelihood of progressing to either HGD or EAC, respectively (FIG. 8). The results can be seen in Table 3. The significance and confidence intervals were Wald based (Table 3).

TABLE 3

| Reference to high risk category | Odds Ratio (95% CI) | | ChiSquare |
|---|---|---|---|
| High vs. Low | 27.8 | (8.00, 132) | 0.0001 |
| High vs. Favourable Intermediate | 10.5 | (3.21, 47.8) | 0.0001 |
| High vs. Unfavourable Intermediate | 4.24 | (1.17, 20.4) | 0.026 |

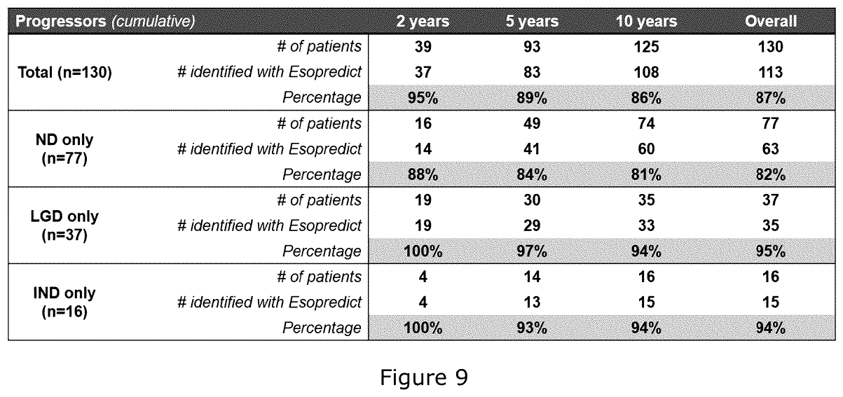

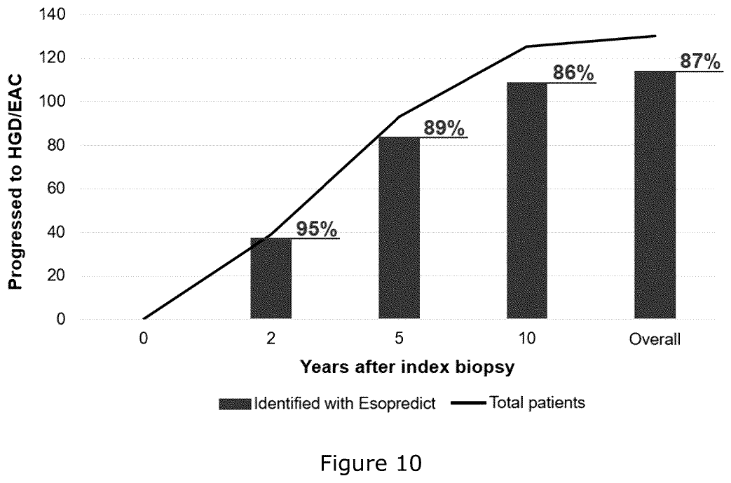

Esopredict was shown to predict progressors based on dysplasia at 2, 5 or 10 years. at an accuracy of 96%, 89% and 86%, respectively, with an overall accuracy of 87%, which included progressors from the entire study cohort (n=130) (FIG. 9 and FIG. 10).

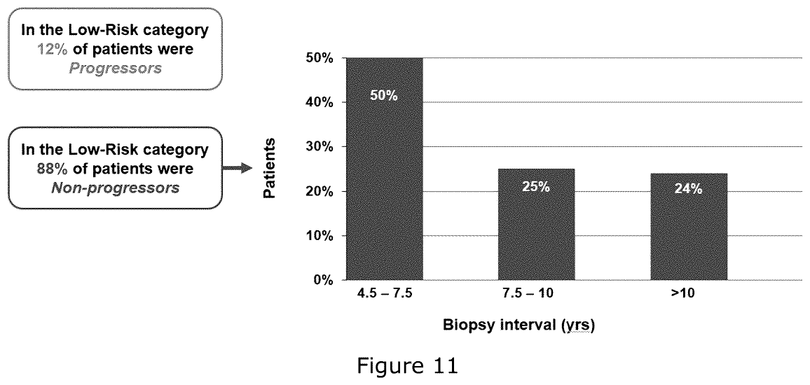

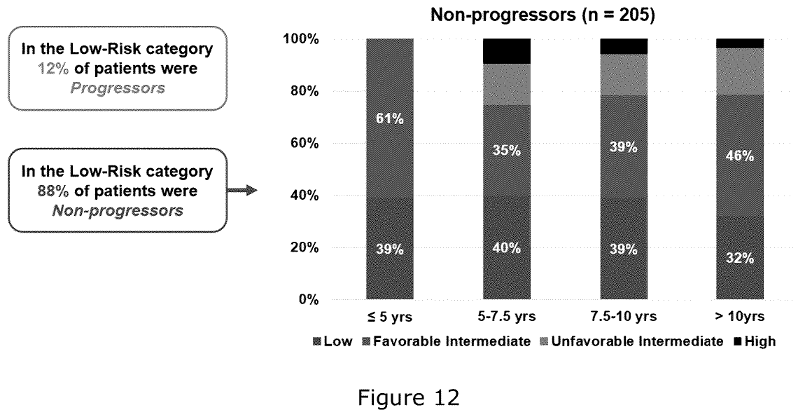

Esopredict was shown to prevent unnecessary surveillance of 88% of patients in the “low risk” category that were non-progressors (n=205), including low-risk patients from entire cohort (n=85) (FIG. 11 and FIG. 12).

### Sensitivity and Specificity

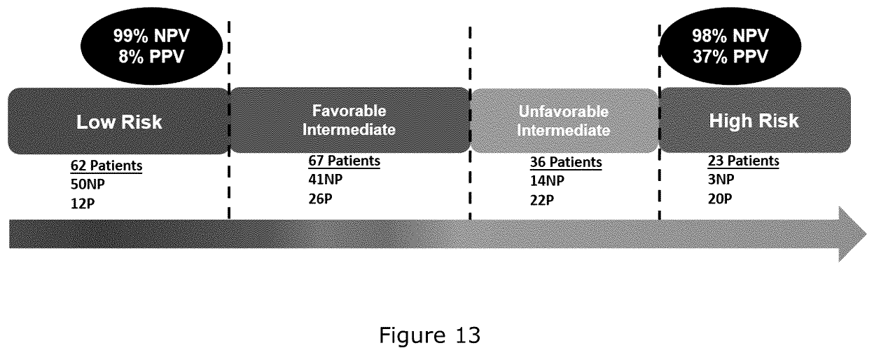

Esopredict was shown to have a 99% NPV and 8% PPV for the low risk category, and a 98% NPV and 37% PPV for the high risk category (FIG. 13).

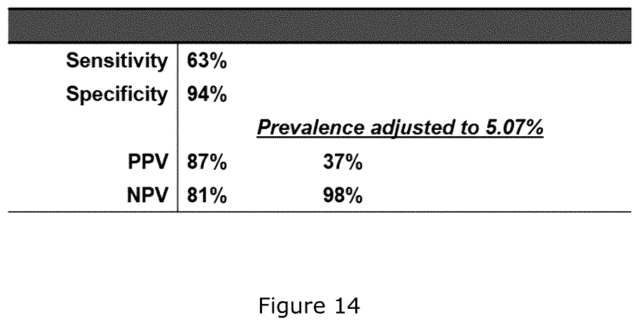

Overall, to have a sensitivity of 63% and a specificity of 94% (FIG. 14).

### Discussion

Predictive biomarkers make surveillance more efficient by focusing on those at highest risk of progression.
Epigenetic biomarkers make surveillance more effective by predicting risk earlier in disease stage.
Biomarkers based on methylation allow prediction of risk earlier in disease stage.
This complements surveillance to more efficiently guide standard of care.
Esopredict allows the risk stratification of neoplastic progression in patients with BE.
Esopredict may help strengthen confidence in current treatment measures of this important disease.
This predictive assay based on epigenetic biomarkers could provide gastroenterologists with valuable information to enhance personalized patient management.

### Example 2—Further Validation

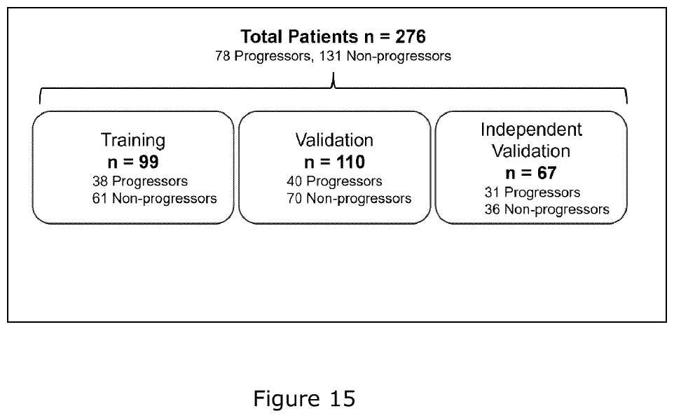

Patient selection: Index biopsies with histological diagnoses of NDBE, IND, or LGD made by an expert GI pathologist at each institution. These index (i.e. baseline) biopopsies were the samples analyzed in this study by the molecular assay and subsequent algorthm. Follow-up biopsy information, which defined patient outcomes was required for the definition of progressor and non-progressor status. Of 209 patients, 78 progressed to HGD/EAC within 5 years (progressors) and 131 had no progression ≥5 years (non-progressors) (FIG. 1). To test our algorithm on patients who progressed at time intervals longer than 5 years, an additional cohort of 31 progressors was independently collected and tested (FIG. 15).

Biopsy procurement and pathologic confirmation: Eight consecutive sections were cut from formalin-fixed paraffin-embedded (FFPE) tissue blocks containing esophageal biopsies from 240 patients with BE (FIG. 15). The histological diagnosies with were collected for both the index biopsies (i.e. assayed) with ND, IND, or LGD and the outcome biopsies (i.e. the last known follow-up biopsy on record) with HGD or EAC. Additionally, patient age, sex, date of biopsies, and any other clinical variables that were obtainable such as BMI, and segment length, smocking status were collected for each patient.

Assay: Esopredict consists of 4 key biomarkers (p16, HPP1, RUNX3, and FBN1) plus age to predict the progression to HGD or EAC within 5 years.

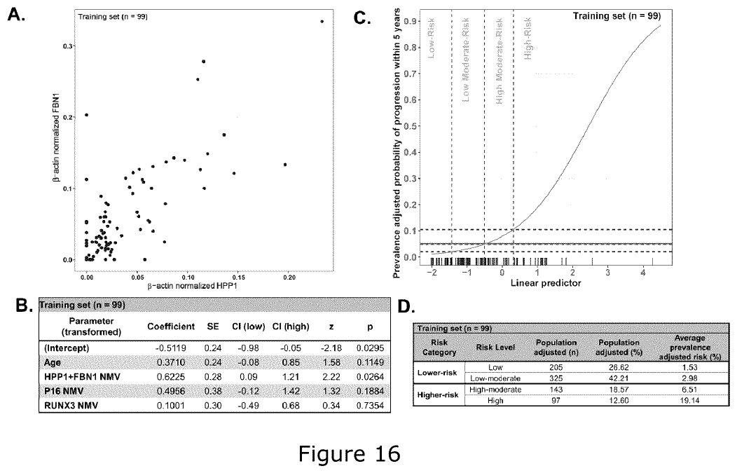

Training cohort and model development: After biomarker selection, clinical samples meeting the specified inclusion criteria were used to build the final model. For model selection and estimation, data conventions were revised using the same parameters for biomarker selection when no amplification was detected (zero) or below the lower limit of quantification (LLOQ, 0.0008). The normalized methylation value (NMV) of each gene was evaluated for performance. Functional form was assessed for each marker. Due to the high correlation between HPP1 and FBN1, an equally weighted average of their NMVs was used to combine the two markers (FIG. 16A). Since p16 showed a biphasic relationship, this marker had a threshold placed at 0.25. Runx3 was not transformed before inclusion in the model. Finally, approximate linearity was achieved for all three predictors via the square root transformation (FIG. 16B). Using the transformed NMV for the 4 genes, a final logistic regression model was estimated with the 4 genes and age.

Validation cohort: Validation was performed by assessing the relationship of the risk score and category and risk level with progression. To obtain confidence intervals for prediction, a logistic model was fit to the validation data using the risk score and adjusted for population prevalence as described previously.

Statistics: To estimate the prevalence-adjusted probability of progression, the intercept term was adjusted using the log odds of the population prevalence. To approximate a predictiveness curve from the training data for the overall population, the sample was assumed to be representative, and non-progressors were repeated to nearly match population prevalence. Under this assumption, the population-adjusted percentiles approximate the population percentiles at each observed score value. Cut-off values were placed to approximate patients that would have a lower-than-average risk of progression based on prevalence (lower-risk category) vs. patients that would have higher-than-average risk (higher-risk category). To further refine these categories, we argeted approximately 27.5% of patients into levels; low-risk, 42% in low moderate-risk, 17.5% in high moderate-risk, and 12.5% in high-risk. These cut-off values were chosen after a visual inspection of the predictiveness curve plotting the risk percentile vs. the predictive probability. Our goal was for the low-risk group to identify a significant number of patients with a clinically meaningful low probability of progression within 5 years (lower risk category). Equally, this logic was applied to have a higher risk category with a clinically meaningful high probability of progression within 5 years. We further refined the categories into risk levels same logic, with the high-risk level to identify a number of patients that would have a clinically meaningful high probability of progression much higher than average, low risk level much lower risk, and intermediate risk levels (low moderate and high moderate) with a probability slightly below or above the average risk of progression. The score was rescaled to the interval [0, 10], with one decimal place, and the cut points were rounded to the nearest value to form the Esopredict score.

### Results

### Development of the Classification Algorithm

Among 240 patients, 209 met our inclusion criteria (described in Methods, Patient selection) and were collected as model training (n=99) and subsequently collected as model validation (n=110) groups from the same six collaborating sites. An additional independent validation (n=33) included patients who progressed but were outside our patient inclusion criteria (>5 years). (FIG. 1). Biomarker and clinical variables were assessed and selected using the Least Absolute Shrinkage and Selection Operator (LASSO) method in a logistic regression setting. An additional independent validation (n=31) was also analyzed to test our assay in progressors with outcome biopsies >5 years (FIG. 15, and FIG. 5).

NMVs of the biomarkers HPP1 and FBN1 were highly correlated when considering bivariate analysis (FIG. 16A), so these biomarkers were combined and averaged, in 1ddition to being transformed. A formula containing the 4 biomarkers plus age was established, and classification accuracy was evaluated using receiver operator criteria (ROC) curves. The Area under the ROC curve (AUC) for this prognostic assay in predicting progression to HGD or EAC was 0.76. Age is correlated with both risks of developing EAC and relatively increased methylation levels, so we compared the assay results to those based on age alone (AUC=0.62). The prevalence-adjusted curve of predicted probability within 5 years for progression to HGD or EAC for the training set is illustrated in FIG. 16C, with the corresponding data table in FIG. 16D.

### Predictive Performance of the Algorithm

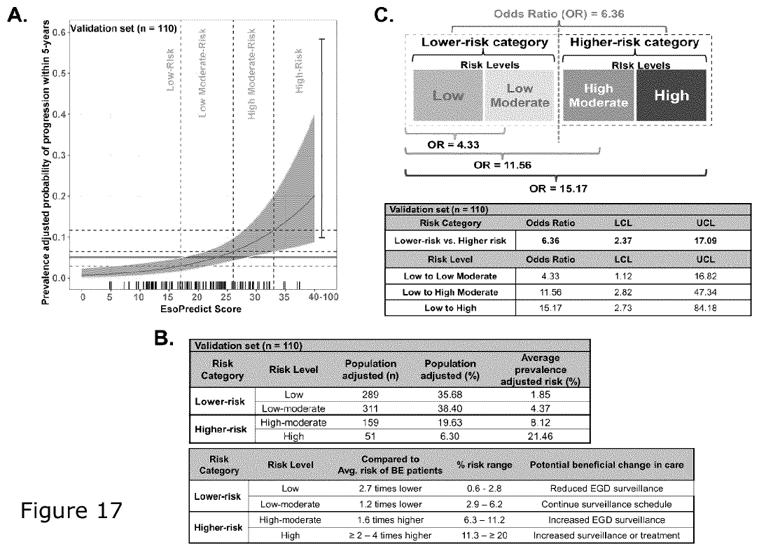

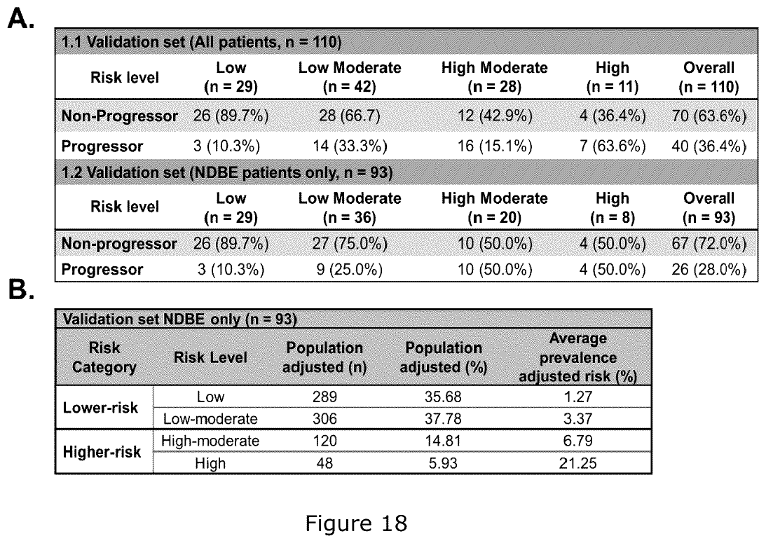

The predicted probability of the validation (n=110) is shown after adjusting for prevalence and population, with each Esopredict score (X-axis) correlating with a specific risk of progression (FIG. 17A). The averages of these parameters within each risk are shown in FIG. 17B. Overall, the odds of progression within 5 years of the higher-risk categories (i.e., high moderate and high risk combined, comprising patients who would likely benefit from improved care management were 6.4 times higher than the progression odds of the lower-risk categories (i.e., low and low moderate-risk combined) (FIG. 17C). Similar trends were observed for the four risk-levels ccomparing the low risk to the other risk levels (FIG. 18C).

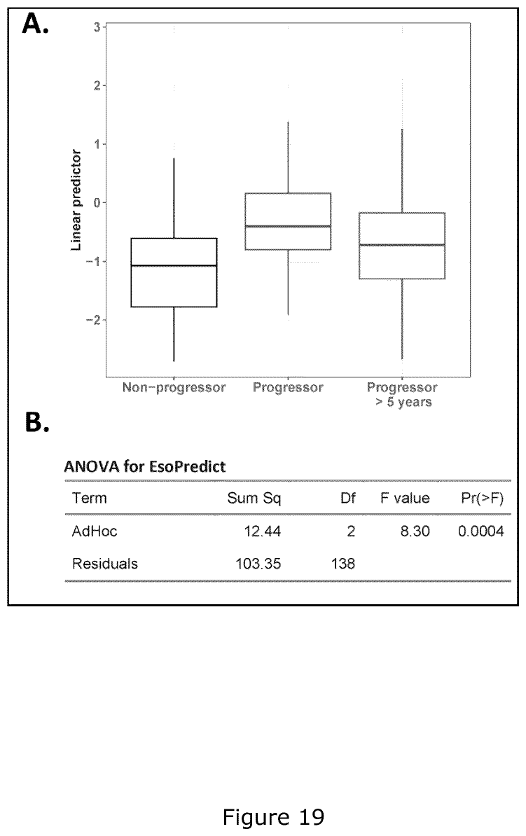

The percentage of progressors was increased in higher risk category (risk levels high moderate and high), while the percentage of non-progressors was increased in lower risk category (risk levels low and low moderate) (FIG. 18A). This trend was consistently observed comparing the full validation set to the subset of patients with index NDBE biopsies (n=93) (FIG. 18A and FIG. 19B).

Additionally, across risk levels, the population-adjusted percentage of NDBE-only patients shifted slightly, with similar average percent risk and population-adjusted percentages (FIG. 18B) compared with the full validation cohort (FIG. 17B). In this validation cohort, 93 patients who had index biopsies of NDBE (85%), aligning closely with the prevalence observed in the general population. This cohort also had 17 patients (15%) whose index (i.e. baseline) biopsies were diagnosed with LGD or IND. Upon analyzing this subset, we identified that 64% (9 out of 14 patients) were categorized in higher risk (high moderate and high risk levels). However, the relatively small size of this subgroup posed challenges for stratifying the analysis by GD or IND analogous to the approach with NBDE cases. An additional validation set included 31 progressors whose outcome was >5 years, with a mean of 6.5 years (5.2-17.8). There was a similar overall trend in increased scores compared to non-progressor patients (FIG. 19), indicating that regardless that progression occurred much longer than a typical surveillance interval (3-5 years), the performance of this assay was comparable to those with follow-up within 5 years, p=0.0004.

### Discussion

This retrospective, multicenter, 240-patient study included 164 patients with index (i.e., initial) biopsies showing NDBE and 76 patients with index biopsies showing IND or LGD. Esopredict provides simplified clinically useful information by stratifying patients as either lower risk category (further refined into low-and low-moderate risk levels) or higher risk category (further refined into high-moderate-and high risk levels). The OR comparing the lower-risk category to the higher-risk category to the lower-risk indicates patients in the latter category are 6.4 times more likely to progress. By further stratifying into four risk levels we gain deeper insights for surveillance and treatment considerations, those clinically relevant levels are defined as; 1) low-risk, with a four times lower-than-average risk of progression (1.85% average), 2) low moderate-risk with a slightly-lower-than-average risk of progression (4.47%), 3) high-moderate risk with a slightly-higher-than-average risk of progression (8.12%), 4) high-risk with more than four times higher-than-average risk of progression (21.46%) (FIG. 17C). We demonstrated that vs. low-risk patients, high-risk patients had 15.2 times higher odds of progression to HGD or EAC within 5 years.

Although progression to HGD or EAC is rare in BE, almost one-quarter of patients who progress do so within 1 year of a negative endoscopy, representing missed opportunities for early intervention. Indeed, 9 patients in our validation (n=110) had an outcome biopsy showing progression at less than 1 year after their index biopsy. Of these patients, 6 were predicted in higher Esopredict risk categories (i.e., high moderate, and high-risk. In our validation cohort (n=110), 85% of patients had an index biopsy of NDBE; among those NDBE patients, 62 had a last-known follow-up biopsy with NDBE-i.e., no change in nondysplastic status at followup intervals ranging from 5 years to 18 years. Esopredict classified 85.0% (n=50) of these 62 patients in the two lower-risk categories (low or low-moderate), with 26 patients being in the low-risk level (i.e., yielding an NPV of 99%), indicating potential decreased surveillance. Among 26 NDBE patients who had progressed based on outcome biopsy within 0-5 years, 14 (57.7%) would have been classified by Esopredict into one of the two higher-risk categories (high moderate or high-risk), indicating the potential to increase surveillance frequency or perform preventative EET. These patients with NDBE would today likely be overlooked based on existing standard-of-care methods. Current evidence suggests that treatment of patients with low-grade dysplasia (LGD) with EET can be a successful low-risk preventative measure; however, concern about over-diagnosis of LGD and care management of the LGD patient population has been controversial. (50, 53, 54) Our validation cohort also included 17 patients (15%) with either IND or LGD index biopsies. Of these patients, 0 had a follow-up biopsy of NDBE, and 0 returned low-risk Esopredict scores. Among these patients, Esopredict classified 11 (64.7%) in higher risk categories (high-moderate or high risk) who had either progressed or remained IND or LGD at the time of outcome biopsy. These results suggest potential clinical utility in managing patient care in BE patients with IND or LGD, warranting additional follow-up studies.

Endoscopic surveillance with histopathologic analysis of biopsy samples is the current standard-of-care method to determine the risk of future neoplasia in BE patients. However, compliance with these programs is not consistently high, with adherence to established guidelines at only 50%. (23-30) Moreover, while underutilization of surveillance may allow preventable cancers to develop, overutilization contributes to higher healthcare costs and inefficient resource utilization. An advantage of the Esopredict assay is that it adds complementary value to the current standard of care, incorporating epigenetic data without the need for additional biopsies to advance the personalized management of patients with BE. More patients that are high-risk and would benefit from preventative EET or increased surveillance can be identified beyond the current method while reducing biopsies in patients at low risk for progression. The low cost, high throughput, and repeatably consistent performance of the biomarkers across multiple development studies make Esopredict an ideal technology for BE surveillance.

Risk stratification of patients diagnosed with BE via Esopredict provides critical information for guiding preventative treatment decisions Using Esopredict, clinicians will be able to use this four-tier risk system with an individualized probability of progression score to determine the most appropriate BE, either lengthen future endoscopic surveillance intervals and avoid EET (low-risk), build confidence around current care management (low moderate-risk), or consider increasing surveillance or EET to prevent the development of one of the most lethal cancers in patients at higher risk.

### REFERENCES

1. Critchley-Thorne RJ, Davison JM, Prichard JW, Reese LM, Zhang Y, Repa K, Li J, Diehl DL, Jhala NC, Ginsberg GG, DeMarshall M, Foxwell T, Jobe BA, Zaidi AH, Duits LC, Bergman JJ, Rustgi A, Falk GW. A Tissue Systems Pathology Test Detects Abnormalities Associated with Prevalent High-Grade Dysplasia and oesophageal Cancer in Barrett's oesophagus. Cancer Epidemiol Biomarkers Prev. 2017 February;26(2):240-248. Epub 2016

2. Critchley-Thorne RJ, Duits LC, Prichard JW, Davison JM, Jobe BA, Campbell BB, Zhang Y, Repa KA, Reese LM, Li J, Diehl DL, Jhala NC, Ginsberg G, DeMarshall M, Foxwell T, Zaidi AH, Lansing Taylor D, Rustgi AK, Bergman JJ, Falk GW. A Tissue Systems Pathology Assay for High-Risk Barrett's oesophagus. Cancer Epidemiol Biomarkers Prev. 2016 June;25(6):958-68. EPI-15-1164. Epub 2016 May 13.

3. Davison JM, Goldblum J, Grewal US, McGrath K, Fasanella K, Deitrick C, DeWard AD, Bossart EA, Hayward SL, Zhang Y, Critchley-Thorne RJ, Thota PN. Independent Blinded Validation of a Tissue Systems Pathology Test to Predict Progression in Patients With Barrett's oesophagus. Am J Gastroenterol. 2020 June;115(6):843-852.

4. Duits LC, Phoa KN, Curvers WL, et al. Barrett's oesophagus patients with low-grade dysplasia can be accurately risk-stratified after histological review by an expert pathology panel. Gut 2015;64(5):700-6.

5. Fitzgerald RC, Di Pietro M, Ragunath K, et al. British Society of Gastroenterology guidelines on the diagnosis and management of Barrett's oesophagus. Gut. 2014;63(1): 7-42.

6. Frei NF, Konte K, Bossart EA, Stebbins K, Zhang Y, Pouw RE, Critchley-Thorne RJ, Bergman JJGHM. Independent Validation of a Tissue Systems Pathology Assay to Predict Future Progression in Nondysplastic Barrett's oesophagus: A Spatial-Temporal Analysis. Clin Transl Gastroenterol. 2020 October;11(10):e00244.

7. Hao J, Critchley-Thorne R, Diehl DL, Snyder SR. A Cost-Effectiveness Analysis Of An Adenocarcinoma Risk Prediction Multi-Biomarker Assay For Patients With Barrett's oesophagus. Clinicoecon Outcomes Res. 2019 Oct. 25;11:623-635.

8. Kahn A, Priyan H, Dierkhising RA, et al. Outcomes of radiofrequency ablation by manual versus self-sizing circumferential balloon catheters for the treatment of dysplastic Barrett's oesophagus: a multicenter comparative cohort study. Gastrointest Endosc 2021;93(4):880-887.

9. Jin Z, Mori Y, Yang J, et al. Hypermethylation of the nel-like 1 gene is a common and early event and is associated with poor prognosis in early-stage oesophageal adenocarcinoma. Oncogene 2007;26(43):6332-40.

10. Jin Z, Olaru A, Yang J, et al. Hypermethylation of tachykinin-1 is a potential biomarker in human oesophageal cancer. Clin Cancer Res 2007;13(21):6293-300.

11. Jin Z, Hamilton JP, Yang J, et al. Hypermethylation of the AKAP12 promoter is a biomarker of Barrett's associated oesophageal neoplastic progression. Cancer Epidemiol Biomarkers Prev 2008;17(1):111-7.

12. Jin Z, Cheng Y, Gu W, et al. A multicenter, double-blinded validation study of methylation biomarkers for progression prediction in Barrett's oesophagus. Cancer Res 2009;69(10):4112-5.

13. Parasa S, Vennalaganti S, Gaddam S, et al. Development and validation of a model to determine risk of progression of Barrett's oesophagus to neoplasia. Gastroenterology. 2018; 154(5): 1282-1289.

14. Sato F, Jin Z, Schulmann K, et al. Three-tiered risk stratification model to predict progression in Barrett's oesophagus using epigenetic and clinical features. PLOS One 2008;3(4):e1890.

15. Schulmann K, Sterian A, Berki A, et al. Inactivation of p16, RUNX3, and HPP1 occurs early in Barrett's associated neoplastic progression and predicts progression risk. Oncogene 2005;24(25):4138-48.

16. Shaheen NJ, Falk GW, Iyer PG, Gerson LB. ACG clinical guideline: diagnosis and management of Barrett's oesophagus. Off J Am Coll Gastroenterol ACG. 2016; 111(1): 30-50.

17. Sharma P, Dent J, Armstrong D, Bergman JJ, Gossner L, Hoshihara Y, Jankowski JA, Junghard O, Lundell L, Tytgat GN, Vieth M. The development and validation of an endoscopic grading system for Barrett's oesophagus: the Prague C & M criteria. Gastroenterology. 2006;131:1392-9. Epub 2006 Aug. 16.

18. Siegel, RL, Miller, KD, Wagle, NS, Jemal, A. Cancer statistics, 2023. CA Cancer J Clin. 2023; 73(1): 17-48.

19. Surveillance E, and End Results (SEER) Program SEER*Stat Database: Mortality—All COD, Aggregated With State, Total U.S. (1969-2019) <Katrina/Rita Population Adjustment>.www.seer.cancer.gov: National Cancer Institute, DCCPS, Surveillance Research Program; 2021.

20. Thrift, A.P. Global burden and epidemiology of Barrett oesophagus and oesophageal cancer. Nat Rev Gastroenterol Hepatol 18, 432-443 (2021).

21. Visrodia K, Singh S, Krishnamoorthi R, Ahlquist DA, Wang KK, Iyer PG, Katzka DA. Magnitude of Missed oesophageal Adenocarcinoma After Barrett's oesophagus Diagnosis: A Systematic Review and Meta-analysis. Gastroenterology. 2016 March; 150(3):599-607.e7; quiz e14-5. Epub 2015 Nov. 24.

22. Wang Z, Kambhampati S, Cheng Y, et al. Methylation Biomarker Panel Performance in EsophaCap Cytology Samples for Diagnosing Barrett's oesophagus: A Prospective Validation Study. Clin Cancer Res 2019; 25(7):2127-2135.

23. Westerveld D, Khullar V, Mramba L, et al. Adherence to quality indicators and surveillance guidelines in the management of Barrett's esophagus: a retrospective analysis. Endosc Int Open 2018;6:E300-E307.

24. Wani S, Williams JL, Komanduri S, et al. Endoscopists systematically undersample patients with long-segment Barrett's esophagus: an analysis of biopsy sampling practices from a quality improvement registry. Gastrointest Endosc 2019;90:732-741 e3.

25. Wani S, Williams JL, Komanduri S, et al. Over-Utilization of Repeat Upper Endoscopy in Patients with Non-dysplastic Barrett's Esophagus: A Quality Registry Study. Am J Gastroenterol 2019;114:1256-1264.

26. Roumans CAM, van der Bogt RD, Steyerberg EW, et al. Adherence to recommendations of Barrett's esophagus surveillance guidelines: a systematic review and meta-analysis. Endoscopy 2020;52:17-28.

27. Ofman JJ, Shaheen NJ, Desai AA, et al. The quality of care in Barrett's esophagus: endoscopist and pathologist practices. Am J Gastroenterol 2001;96:876-81.

28. Dalal KS, Coffing J, Imperiale TF. Adherence to Surveillance Guidelines in Nondysplastic Barrett's Esophagus. J Clin Gastroenterol 2018;52:217-222.

29. Cruz JD, Paculdo D, Ganesan D, et al. Clinical variation in surveillance and management of Barrett's esophagus: A cross-sectional study of gastroenterologists and gastrointestinal surgeons. Medicine (Baltimore) 2022;101:e32187.

30. Abrams JA, Kapel RC, Lindberg GM, et al. Adherence to biopsy guidelines for Barrett's esophagus surveillance in the community setting in the United States. Clin Gastroenterol Hepatol 2009;7:736-42; quiz 710.

1. A method for detecting biomarkers associated with progression of Barrett's oesophagus in a subject, comprising:
a) providing a cell sample from oesophagus of the subject;
b) detecting methylation one or more nucleic acid biomarkers in the sample using a biochemical assay;
c) using a computer algorithm, determining a parameter associated with the detected in step b) methylation of the one or more biomarkers;
d) comparing the parameter calculated in step c) to at least one predetermined cut-off value, wherein the comparison is indicative of risk of Barrett's oesophagus progression in the subject; and
e) providing a computer-generated output based on the comparison.

2. The method of claim 1, wherein the methylation of the one or more biomarkers in step b) is detected using DNA methylation analysis.

3. The method of claim 2, wherein step (b) comprises performing a polymerase chain reaction (PCR)-based technique.

4. The method of claim 3, wherein the PCR-based technique is selected from quantitative methylation specific PCR (QMSP), digital methylation specific PCR (dPCR), or droplet digital methylation specific PCR (ddPCR).

5. The method of claim 1, wherein the one or more biomarkers include methylation in a gene.

6. The method of claim 5, wherein the one or more biomarkers comprises one or more genes comprising P16, RUNX3, HPP1 and FBN1.

7. The method of claim 6, wherein the one or more further comprises one or more genes comprising p53, mVIM, mCCNA1, TAC1, NELL1, AKAP12, SST, ABCB1, BMP3, COL23A1, FADS1 and PRDM2.

8. The method of claim 1, wherein, in step c), with the computer algorithm utilizes one or more further clinical factors associated with the subject.

9. The method of claim 8, wherein the one or more further clinical factors associated the subject include age of the subject.

10. The method of claim 1, wherein the computer algorithm is a trained algorithm.

11. The method of claim 10, wherein the computer algorithm is a least absolute shrinkage and selection operator (LASSO) algorithm.

12. The method of claim 10, wherein the algorithm is a logistic regression algorithm.

13. The method of claim 1, wherein the sample comprises one or more cells from surface of the oesophagus.

14. The method of claim 13, wherein the sample is a non-endoscopic sample.

15. The method of claim 14, wherein the sample is provided by oesophageal brushing, a swallowable sponge device, or by retrieving a swallowable device from the subject that has been swallowed by the subject, wherein the device comprises an abrasive material configured to collect cells.

16. The method of claim 1, wherein the subject is human.

17. The method of claim 1, wherein the subject is diagnosed with Barrett's oesophagus characterised as having no dysplasia, reactive atypia, indefinite for dysplasia, low grade dysplasia, or high grade dysplasia.

18. The method of claim 1, wherein the subject is diagnosed with Barrett's oesophagus characterised by Prague stage of at least C1, at least M1, at least C2, at least M2, at least C3, at least M3, at least C1 or M3, or any combination thereof.

19. The method of claim 1, wherein the subject has, is suspected of having, or has been identified as being at risk of developing, oesophageal cancer.

20. The method of claim 1, wherein the subject has one or more risk factors for oesophageal cancer and/or Barrett's oesophagus, optionally selected from:
a) being age 55 or over;
b) being a man;
c) being a smoker;
d) being an alcohol drinker;
e) having gastroesophageal reflux disease;
f) being obese;
g) suffering from achalasia;
h) having a history of certain other cancers; and/or
i) suffering from Tylosis or Plummer-Vinson syndrome.

21. The method of claim 1, wherein the risk of progression of Barrett's oesophagus is a risk of progressing to dysplasia or oesophageal cancer.

22. The method of claim 21, wherein oesophageal cancer is oesophageal adenocarcinoma.

23. The method of claim 1, wherein the output comprises a risk level associated with progression to dysplasia and/or oesophageal adenocarcinoma.

24. The method of claim 1, wherein the output comprises a risk categorisation assigned to the subject.

25. The method of claim 24, wherein during step e) the subject is assigned a “high risk” of progression if the parameter calculated in step d) is equal to or above an upper predetermined cut-off value.

26. The method of claim 25, wherein during step e) the subject is assigned a “low risk” of progression if the parameter calculated in step d) is below a lower predetermined cut-off value,
wherein the lower predetermined cut-off value is lower than the upper predetermined cut-off value.

27. The method of claim 26, wherein during step e) the subject is assigned an “intermediate risk” of progression if the parameter calculated in step d) is below the upper predetermined cut-off value and equal to or above the lower predetermined cut-off value.

28. The method of claim 27, wherein during step e) the subject is assigned an “unfavourable intermediate risk” of progression if the parameter calculated in step d) is below the upper predetermined cut-off value and equal to or above an intermediate predetermined cut-off value, and/or assigned a “favourable intermediate risk” of progression if the parameter calculated in step d) is below the intermediate predetermined cut-off value and equal to or above the lower predetermined cut-off value,
wherein the intermediate predetermined cut-off value is between the upper and lower predetermined cut-off values.

29. A method for detecting biomarkers associated with progression of Barrett's oesophagus in a subject and treating the subject, comprising:
a) providing a cell sample from oesophagus of the subject;
b) detecting methylation one or more nucleic acid biomarkers in the sample using a biochemical assay;
c) using a computer algorithm, determining a parameter associated with the detected in step b) methylation of the one or more biomarkers;
d) comparing the parameter calculated in step c) to at least one predetermined cut-off value, wherein the comparison is indicative of risk of Barrett's oesophagus progression in the subject; and
e) performing Barrett's oesophagus treatment on the subject.

30. The method of claim 29, wherein the Barrett's oesophagus treatment comprises one or more of endoscopy, an NSAID, a PPL, an anti-cancer agent, endoscopic resection, endoscopic ablation, or radiotherapy.

31. A system for determining a risk of progression of Barrett's oesophagus in a subject, comprising:
a) a station for, using a biochemical assay, detecting methylation one or more nucleic acid biomarkers in a sample obtained from the subject;
c) a computer running an algorithm for:
1) determining a parameter associated with the detected in step b) methylation of the one or more biomarkers; and
2) comparing the parameter calculated in step c) to at least one predetermined cut-off value, wherein the comparison is indicative of risk of Barrett's oesophagus progression in the subject.
